(19) United States
(12) Patent Application Publication
(10) Pub. No.: US 2022/0296852 A1
(43) Pub. Date: Sep. 22, 2022

(54) MAMMALIAN BODY IMPLANTABLE FLUID FLOW INFLUENCING DEVICE

(71) Applicant: Puzzle Medical Devices Inc., Montreal (CA)

(72) Inventor: Gabriel GEORGES, Quebec (CA)

(73) Assignee: Puzzle Medical Devices Inc., Montreal (CA)

(21) Appl. No.: 17/712,700

(22) Filed: Apr. 4, 2022

Related U.S. Application Data
(63) Continuation of application No. PCT/IB2021/052925, filed on Apr. 8, 2021.
Application No. PCT/IB2021/052925 is a continuation of application No. PCT/CA2020/051673, filed on Dec. 4, 2020.
Application No. PCT/CA2020/051673 is a continuation-in-part of application No. 17/062,616, filed on Oct. 4, 2020, now abandoned.
Application No. 17/062,616 is a continuation-in-part of application No. PCT/CA2020/051673, filed on Dec. 4, 2020.
(60) Provisional application No. 62/910,830, filed on Oct. 4, 2019.
(60) Provisional application No. 63/110,871, filed on Nov. 6, 2020.
(60) Provisional application No. 62/910,830, filed on Oct. 4, 2019.

(51) Int. Cl. A61M 25/00; A61F 2/24; A61F 2/90

(52) U.S. Cl. CPC A61M 25/0029; A61F 2/2418; A61F 2/2436; A61F 2/90; A61M 25/0012; A61F 2220/0008; A61B 2017/00606

(57) ABSTRACT

Mammalian body implantable fluid flow influencing device for influencing flow of a first fluid within a first bodily conduit via a flow of a second fluid within a second bodily conduit, comprising a first and a second working end. Each end has a vaned rotor and an anchor for anchoring that end within a bodily conduit. Each end having a delivery configuration for percutaneous transcatheter endovascular delivery to an implantation site within a bodily conduit. A driveshaft assembly operatively interconnects the second end rotor with the first end rotor to transmit rotational movement of the second rotor to the first rotor. When the device is implanted within the body, the second vaned rotor acts as a turbine and extracts kinetic energy from the second fluid, and the first vaned rotor acts an impeller and imparts kinetic energy to the first fluid. The device is motorless.

### CROSS-REFERENCE

The present application is a continuation of International Patent Application No. PCT/IB2021/052925, filed Apr. 8, 2021 (pending) (the '925 PCT), which is a continuation of International Patent Application No. PCT/CA2020/051673, filed Dec. 4, 2020 (pending) (the '673 PCT), which claims priority to and the benefit of U.S. Provisional Application No. 62/910,830, filed Oct. 4, 2019 (expired) (the '830 Provisional). The '925 PCT also claims priority to and the benefit of U.S. Provisional Application No. 63/110,871, filed Nov. 6, 2020 (expired) and is a continuation-in-part of U.S. application Ser. No. 17/062,616, filed Oct. 4, 2020, (abandoned), which claims priority to and the benefit of the '830 provisional. The present application is also a continuation-in-part of the '673 PCT, which claims priority to and the benefit of the '830 Provisional. The contents of each one of the foregoing applications are incorporated herein by reference in their entirety for all purposes.

### FIELD

The present technology relates to mammalian body implantable fluid flow influencing devices.

### BACKGROUND

### General

Fluid carrying conduits in patients, such as blood vessels or other conduits near the heart, liver or kidneys that carry fluids other than blood (e.g., urine, lymph, etc.), may require fluid flow influencing (e.g., an increase in fluid flow rate, a decrease in fluid flow rate, a stoppage of fluid flow, a diversion of fluid flow, etc.) in various medical situations.

### Heart Failure

Heart failure is an example of a common such situation. In patients with heart failure, their heart becomes unable to pump enough blood to meet their body's needs for blood and oxygen.

Heart failure is a disease affecting upwards of 6 million Americans and 26 million people worldwide at any given time. There is no cure. For those suffering from heart failure, their ability to function in everyday life and their overall quality of life steadily and inevitably decline. There may be times of rapid deterioration. Even with the best of medical care, heart failure sufferers' symptoms will slowly, inevitably progress. They will rapidly become limited in their activities. At some point in time, they will experience increasing symptoms of the disease even at rest and under optimal medical therapy. People with end-stage heart failure disease currently have a 2-year estimated chance of survival of only 20%.

To try to improve this somber forecast of the probable course and outcome of the disease, multiple strategies for caring for people having heart disease have been developed. Such strategies include both short-term mechanical patient support options, as well as longer-term patient support options. Unfortunately, none of the options currently available are optimal.

Open Surgery Vs. Minimally Invasive Surgery

Prior to review of the current conventional heart failure treatment possibilities, it should be noted that all such treatments are surgical in nature. They may be carried out on a patient suffering from the disease either via “open surgery” (i.e., the traditional surgical method of the cutting of skin and tissues so that the surgeon has a full view of the structures or organs involved) or via “minimally invasive surgery” (i.e., newer surgical techniques that do not require large incisions). Examples of minimally invasive surgical techniques are percutaneous transcatheter techniques, in which a catheter (e.g., a relatively long flexible tube) is inserted into the patient's body and the intervention is performed through the lumen (i.e., the hollow cavity) of the catheter at a site distal to (e.g., away from) the catheter insertion site. As compared with open surgical techniques, transcatheter techniques generally are lower risk to the patient, shorter in time for the surgeon to perform, and have shorter patient recuperation times. They are usually preferred by patients.

### Heart Transplants

One current treatment possibility for heart failure is a heart transplant. Heart transplantation involves the removal of a patient's diseased heart and its replacement with a healthier heart from a heart donor. There are, however, an extremely limited number of donor hearts available. In North America for example, only about 3,000 donor hearts are available each year. So, heart transplantation is not an option which is generally available to patients, as the number of donor hearts is far less than the number of sufferers of the disease. Further, heart transplantation obviously requires very invasive open surgery. It carries additional significant risks, including (but in no way limited to) transplant coronary artery disease and life-long suppression of the recipient's immune system. For all of these reasons, heart transplantation is in most cases limited to younger patients, and therefore younger patients are prioritized on heart transplant lists.

### Artificial Hearts

Another current treatment possibility for heart disease is through the removal of a patient's diseased heart and its replacement with an artificial heart device (typically known as a “total artificial heart”). While the number of total artificial hearts is not limited (as is the case with donor human hearts) as they are manufactured devices, at the moment their use is limited to being only temporary. No total artificial heart is available for permanent implantation. Thus, total artificial hearts are used in patients who are in the end-stages of heart failure disease, but for whom no donor heart is yet available. Their use is quite limited, as the number of donor hearts is limited. In addition, implantation of a total artificial heart still requires very invasive open surgery, and carries risks as noted above. There are very few total artificial heart products currently available for use in patients. One product is the SynCardia™ Temporary Artificial Heart. Another potential product, which is still in development, is the Carmat™ artificial heart.

### Ventricular Assist Devices (Open Surgical Implantation)

A third current treatment possibility for heart disease, and the most common, is through the implantation and use of what is known as a “Ventricular Assist Device” (commonly abbreviated to and referred to as a ‘VAD’). A VAD is a mechanical pump that is surgically implanted within a patient to help a weakened heart pump blood. Unlike a total artificial heart, a VAD does not replace a patient's own heart, instead it helps the patient's native heart pump blood. VADs may be used to help the left side of a patient's heart, in which case they are known as LVADs. Or, they may be used to help the right side of a patient's heart, in which case they are known as RVADs. LVADs are far more commonly used as left heart failure disease is faire more common. Currently, VADs may either be used as a bridge until a heart transplant can be performed (as is the case with total artificial hearts) or they may be used long term in patients whose condition makes it impossible to receive a heart transplant or who require immediate long-term support. There are different types and configurations of VADs, some of which will be discussed below.

Common to almost all currently available VADs is that their implantation requires open surgery, and carries the downsides and risks thereof noted above, and others. The complication rate and the mortality rate associated with the use of VADs are both significant. For example, patients are at risk of embolic stroke (e.g., a stroke caused by the blockage of a blood vessel due to a blood clot having formed), for amongst other reasons, the positioning of a VAD at the apex of the heart. Patients are also at risk of a cerebral (i.e., brain) or gastro-intestinal hemorrhage as most VADs pump blood continuously (as opposed to a normal heart, which pumps blood in pulses). This continuous pumping of blood causes the patient's blood vessels to become more fragile (and thus prone to hemorrhaging) and also causes a decrease in the patent's von Willebrand factor (which is a molecule in human blood that is part of the process to prevent and stop bleeding). Further, owing to the complexity of the VAD implantation surgery, VADs are only implanted in specialized centers. Indeed, the number one reason for patients refusing to undergo VAD implantation is the patient's fear of such invasive implantation surgery and the complications arising therefrom. For all of these reasons, although more than 250,000 heart disease suffers in North America alone could benefit from VAD implantation, there are less than 4,000 yearly VAD implants in the United States.

In terms of types and configurations of VADs, multiple generations of VADs were developed over the past few decades. The following discussion of such generations is not intended to be exhaustive but merely exemplary.

The first-generation of VADs were membrane-based and provided pulsatile flow (e.g., Thoratec™ PVAD, IVAD, Heartmate™ XVE, Heartmate™ IP1000 and VE, WorldHeart™ Novacor™ and Arrow International LionHeart™ LVD2000). Some of the major disadvantages of first-generation VAD's were their high energy requirements, their large size (which complicated surgical implantation), and their limited durability.

Second-generation VADs featured continuous axial flow pumps. These devices were smaller and featured fewer moving parts, which resulted in an overall better design than their first-generation predecessors. The internal rotor of the second-generation VADs were suspended on contact bearings which created high shear stress zones at risk of thrombus formation and hemolysis. The Thoratec™ Heartmate™ II was the most widely used VAD in its class. Other examples of second-generation VADs include the Jarvik Heart Jarvik™ 2000 and the MicroMed™ Heart Assist 5.

Third-generation VADs have all of the advantages of the second-generation VADs (over the first-generation ones). And, they featured non-contact magnetic levitation of the centrifugal rotor, which reduces overall shear stress generated by the pump. They are thus less prone to thrombus formation and hemolysis compared to second-generation devices. Currently available third-generation VADs include the Terumo™ DuraHeart™, the Medtronic™ Heartware™ HVAD and the Abbott™ Heartmate™ III.

All of these generations of VADs described above that are currently in use (or previously had been used) require (or required) invasive classic open surgery (e.g., a median sternotomy or a less invasive mini-thoracotomy). During the implantation procedure, a VAD is surgically attached (e.g., sutured) to the heart while the main VAD body remains external to the patient's vasculature (e.g., heart and blood vessels). The pump inlet of the VAD is sutured to the left or right ventricle of the heart (depending on whether the VAD is an LVAD or an RVAD) and the outflow tubing from the VAD is sutured to the aorta (in the case of an LVAD) or the pulmonary artery (in the case of an RVAD).

### Ventricular Assist Devices (Minimally Invasive Surgical Implantation)

As was described above, however, patients prefer minimally invasive percutaneous transcatheter interventions to open surgery. And thus, the most recent efforts in the development of mechanical support strategies for people with heart disease have been made towards the development of pumps that do not require open surgery, but rather could be implantable transcatheter.

Currently, the only commercial product that can be implanted percutaneously transcatheter is the Impella™ family of micro-pump devices from Abiomed™. An Impella device has a single micro axial pump (e.g., having an impeller) with a cannula (e.g., a small tube-like structure). The device is implanted within the left ventricle (in the case of, e.g., the Impella CP LVAD) of the heart so as to cross the aortic valve, or in the inferior vena cava (in the case of the Impella RP RVAD) so as to pass through the right ventricle and cross the tricuspid and pulmonary valve. Thus, the inlet of the pump is within the ventricle or within the vessels that discharge fluid into the ventricle and the outlet of the pump is outside of the heart, in the aorta (in the case of the Impella CP LVAD) and in the pulmonary artery (in the case of the Impella RP RVAD). As the pump impeller turns, blood is drawn into the device through the pump inlet. The blood then travels under pressure having been imparted by the pump through the cannula and exits the device through the pump outlet in the aorta or pulmonary artery (as the case may be). In this manner, the VAD provides pumping assistance to the ventricle of the heart.

An Impella device is implanted via a percutaneous procedure. In a percutaneous procedure access to the patient's internal organs is made via needle-puncture of the skin (e.g., via the well-known conventional Seldinger technique). Typically, in such procedures, the needle-puncture site is relatively remote from the actual internal organs that the surgeon will be operating on. For example, although it is the heart that a surgeon will be operating on, the initial needle puncture of the skin takes place in the patient's groin area so that the surgeon can access the patient's vasculature through the femoral vessels (such a procedure is thus termed “endovascular”). Once access is obtained, the surgeon can advance the necessary equipment to conduct the surgical procedure through the patient's vasculature to their heart. The surgeon then conducts the procedure on the heart, usually via wires extending from the equipment, travelling through the patient's vasculature and outside of the patient's body via the access opening that the surgeon had previously made. Once the procedure has been completed, the surgeon removes whatever equipment needs to be removed from the patient's vasculature in the same manner. In such procedures, access via the femoral artery (in the patient's groin area) or the axillary artery (about the patient's clavicle) are more common.

Two drawbacks of an Impella type left heart device leading to potential harm are damage to the aortic valve (as the pump body crosses the aortic valve from the left ventricle to the aorta), and in-use device movement (as the device is unanchored when in use). These drawbacks prevent an Impella type device from being used as an outpatient solution. Such devices must be used in a clinical setting. Thus, the Abiomed™ Impella™ pump device is approved for short term support in cardiogenic shock or high-risk percutaneous coronary interventions.

Modular Ventricular Assist Devices (Minimally Invasive Surgical Implantation)

In commonly-owned Int'l. Pat. App. Pub. No. WO 2020/198765 A2 (Puzzle Medical Devices Inc.), published Oct. 1, 2020, entitled “Modular Mammalian Body Implantable Fluid Flow Influencing Device and Related Methods” (hereinafter the “WO '765 Publication”), we proposed a modular ventricular assist device believed to be an improvement over the Impella type device (at least in one respect). One potential benefit of such modular ventricular assist devices is that they may in the future be used in chronic care situations, by reducing blood damage related complications (amongst other advantages), allowing the patient to leave the hospital with the device implanted. Further development in this respect is desirable, as currently there are no percutaneously transcatherly implantable VADs that are available for chronic use situations. (The entire contents of the WO '765 Publication are incorporated herein by reference for all purposes.)

### Right Heart Failure Following Implantation of an LVAD

As was mentioned above, LVADs (of whatever type) are far more commonly used, in both acute care and chronic care situations, as failure of the left side of the heart (commonly called “left heart failure” or “LHF”) occurs far more often, at least at first. At least at first because as left heart failure in a patient progresses (e.g., gets worse) the left heart filing pressures rise, which leads to an increase in lung and pulmonary artery pressures (as the left heart pumps blood oxygenated blood received from the lungs to the rest of the body). This in turn leads to increased resistance and right ventricle load (as the right heart pumps deoxygenated blood from the body to the lungs). This situation is known as “pulmonary hypertension” or “PH”. Pulmonary hypertension leads to right heart failure, and it is observed in 60% to 83% of patients with left heart failure. Pulmonary hypertension is a predictor of all-cause mortality in these patients, with each 5 mm Hg rise in right ventricular systolic pressure (RVSP) being associated with a 6% increased risk of death.

In addition, right heart failure, especially in patients with pulmonary hypertension, is a common complication following LVAD implantation because the right heart suddenly needs to match the output of the left heart, which is now assisted by the LVAD. Right heart failure thus complicates 20-50% of LVAD implantation cases and is a major driver of post-operative mortality in LVAD implantation patients.

### Current RHF Following LVAD Implantation Treatment Techniques

Various methods have been proposed and tried to improve this situation. Examples of such methods include, attaching the patient to an extracorporeal membrane oxygenation (ECMO) machine, implanting a standard RVAD, or implanting a specialized system such as a Tandemlife™ ProtekDuo™. (For a more fulsome discussion of these methods, the reader is referred to: “Right ventricular failure after left ventricular assistance device implantation: a review of the literature”, Valeria LO COCO et al., J. Thorac. Dis. 2021; 13(2):1256-1269|http://dx.doi.org/10.21037/jtd-20-2228 (Vol. 13, No. 2, Feb. 2, 2021), which is incorporated herein by reference in its entirety for all purposes.)

None of these methods are optimal, however. Common to all of them is that they require additional, usually complicated surgery, to implant a second device (in addition to the LVAD) into the patient; that second device being itself connected to an apparatus outside of the patient, apart from whatever apparatus the LVAD itself is connected to. Implantation of such a second device increases the risk to the patient. Moreover, none of these aforementioned devices are suitable for long term use as the patient cannot leave the hospital while they are implanted. Out of all of the currently available RHF ameliorative options when an LVAD is implanted, the Impella RP, which is percutaneously endovascularly transcatherly implanted through venous access, may be the least invasive option available. However, the Impella RP is not intended for ambulatory nor chronic use (as it is prone to displacement because it is not anchored within the vasculature).

Improved ameliorative devices (and other similar body fluid flow influencing devices) would certainly be advantageous, as there are many drawbacks with current such devices and methods.

### Impellers

Both the Impella type devices and the modular VAD of the '765 Publication use axial impellers. An impeller is a mechanical device that employs a rotating rotor to impart energy to a fluid causing it to flow (or increasing its flow rate). The rotor is caused to rotate by an external source of energy, e.g., an electrical motor. Typically, modern impellers employ vaned rotors; the rotating rotor vanes act on the fluid imparting energy to it causing it to flow (or increasing its rate of flow) while expending the externally-sourced energy in the process. (“Vaned rotors” are also known in the art as “bladed rotors”, and “vanes” are known in the art as “blades”. In the context of the present specification these terms should be understood as synonyms, although for solely for the purposes of consistency, only expressions using “vane” have been used in this document.). An axial impeller is an impeller that is designed to move fluid in a direction parallel to its longitudinal axis.

### Turbines

The “opposite” of an impeller is a turbine. Turbines are mechanical devices that employ rotors to extract energy from a flowing fluid (e.g., a gas or a liquid) and convert that energy into useful work. Typically, modern turbines employ vaned rotors; the flowing fluid acts on the vanes to cause the rotor to rotate, imparting the energy to the rotor to cause rotational movement, with the fluid losing that energy in the process. Commonly found real-life turbines are windmills and waterwheels. These structures, which have been around since ancient times, were historically employed to use the energy of moving wind or water to do mechanical work (e.g., grinding grain into flour). Today, they are frequently used to take that energy and generate electricity.

International Patent Application Publication No. WO 2017/217946 A1

Int'l Pat. App. Pub. No. WO 2017/217946 A1, published Dec. 21, 2017, entitled “Self-Propeller Venous Blood Pump”, (hereinafter the “WO '946 Publication”), purports to describe: “ . . . a surgically implantable self-driven ventricle assist device (blood pump) for use in staged partial or total cavopulmonary connection. Total cavopulmonary connection is the main surgical intervention used to treat children born with univentricular congenital heart defects. The invention comprises an aortic turbine that uses some systemic blood from the left ventricle as an energy source and a venous pump that is coupled magnetically or mechanically to said turbine. (Technical Field . . . , the WO '946 Publication).

To facilitate understanding of the device described in the WO '946 Publication, excerpts from the detailed description of the WO '946 Publication are reproduced hereinbelow, with reference to FIG. 1 of the WO '946 Publication, which is reproduced herein as FIG. 1.

“FIG. 1 illustrates the placement of . . . a cavopulmonary assist device (10). Cavopulmonary assist device (10) comprises pump unit (20) and turbine unit (30) coupled by coupling (40). Coupling can be implanted external to the cardiovascular system and also represent and maintain the tissue interface of pulmonary and aortic vessel walls and other tissues. It can also be utilized to support the intended system in the chest cavity for permanent use.” (Detailed Description . . . , pgs. 7-8, the WO '946 Publication).

“Cavopulmonary assist device (10) is designed primarily for patients with Fontan circulation with total cavopulmonary connection (TCPC), namely, patients who have undergone superior vena cava pulmonary artery anastomosis and inferior vena cava-pulmonary artery bridging via a composite conduit in an approximately “+” shape junction. Pump unit (20) is operationally connected to inferior vena cava (2) via an artificial or tissue engineered conduit (9) and the junction of superior vena cava (3) and right (4) and left (5) pulmonary arteries directly . . . . Deoxygenated blood coming from inferior vena cava (2) is pumped into right (4) and left (5) pulmonary arteries by pump unit (20), while deoxygenated blood coming from superior vena cava (3) flows into right (4) and left (5) pulmonary arteries by gravity . . . ” (Detailed Description . . . , pg. 8, the WO '946 Publication).

“Pump unit (20) is not dependent on an external power source. Rotation required by pump unit (20) is generated by turbine unit (30) and transmitted to pump unit (20) by coupling (40) . . . . Preferably, coupling (40) is a mechanical coupling, such as a shaft. Coupling (40) can also be made flexible material for non-invasive delivery of the entire unit to the body.” (Detailed Description . . . , pg. 8, the WO '946 Publication).

“Turbine unit (30) uses a fraction (10-20% of overall 5 L/min) of the systemic blood flow that is supplied by the single ventricle heart (1) and transmits this rotation to pump unit (20). Blood outflow is directed to downstream vasculature, such as an artery or vein to rejoin systemic circulation. The fraction of systemic blood used has negligible effect on systemic circulation flowrate and pressure gradient.” (Detailed Description . . . , pg. 9, the WO '946 Publication).

The device described in the WO '946 Publication is particular for use in young patents with functionally univentricular congenital heart defects who undergo a particular surgical procedure known as Fontan circulation. It is not at all appropriate for older patients suffering from heart failure.

### SUMMARY

It is thus an object of the present technology to ameliorate at least one of the inconveniences present in the prior art, be it one of those described hereinabove or another.

It is a further object of the present technology to provide an improved fluid flow influencing device, at least as compared with one prior art device, be it one of those described hereinabove or another, and method of use of such device.

The present technology results (at least in part) from the developer's efforts to develop an improved device that could be usable to ameliorate a situation where a patient is a risk of development of right heart failure following the implantation of an LVAD, but that could avoid (or at least minimize the impact of) at least one of the problems of prior art devices.

The present technology is based (at least in part) on the principle that a flowing fluid has kinetic energy that can be extracted, transferred, and used to do work. This principle, which is commonly put to use outside of a living body (as described above), can be put to use inside in a mammalian (e.g., human) body, specifically through the transfer of the kinetic energy of one fluid flow to another fluid flow. In practical terms, at a high level, the present technology provides (at least in some implementations) a relatively simple minimally-invasively implantable device capable of harnessing the flow of fluid in one body conduit (e.g., blood in one artery) as a energy source to increase the rate of flow of fluid in another body conduit (e.g., blood in a different artery). This can be achieved, at least in one implementation of the present technology, by having a turbine in the first fluid flow operatively connected to an impeller in the second fluid flow.

Further, in some implementations of the present technology, the kinetic energy being extracted from the fluid flow by the turbine does not need to have been produced by the mammalian body itself (e.g., energy having been produced by that body's heart pumping blood (although this too is certainly possible)). Such kinetic energy could have been imparted to the fluid flow by an electromechanical device upstream of the turbine (e.g., an electromechanical device capable of converting electrical energy into mechanical energy). Thus, for example, in the case where an LVAD is implanted into a patient, (at a high level) the electrical energy supplied to the LVAD (e.g., from a battery or some other power source) is converted by the LVAD's pumping unit into the kinetic energy of the increased blood flow flowing out of the pump. Downstream of the pump outlet, some of this kinetic energy can be extracted from the flowing blood by a turbine of a device being an implementation of the present technology and then used to drive an impeller of that device having been implanted in another conduit of that body. The impeller then transfers that energy into increased kinetic energy of fluid flowing in that second conduit increasing its flow rate.

In such a system, depending on the specifics of the implementation, a device of the present technology could be used to alleviate right heart failure following the implantation of LVAD, in an improved manner over a prior art device. For example, a device of the present technology could be implanted percutaneously transcatheterly and could not require additional wires to be connected to an apparatus external to the patient. Thus, in one implementation, a device of the present technology (that is designed for use (and authorized for use) in chronic care situations) could be used in combination with an LVAD that is designed for use (and authorized for use) in chronic care situations, allowing a patient having both devices implanted in them to be ambulatory and leave the hospital.

It should be understood, however, that although devices of the present technology were developed with an objective of use in patients with heart failure, such devices are not restricted to such uses. In various implementations, devices of the present technology may be used to assist with other bodily conditions, fluids and conduits, whether they involve blood flow and/or the vasculature or not.

Thus, in one aspect, the present technology provides a mammalian body intralumenal implantable fluid flow influencing device for influencing flow of a first fluid within a first conduit of the mammalian body via a flow of a second fluid within a second conduit of the mammalian body. The device comprises a first working end, a second working end, and a driveshaft assembly. The first working end has a first vaned rotor and a first anchor for anchoring the first working end within the first conduit. The first working end has a delivery configuration in which it is dimensioned and shaped to be delivered to a first implantation site within the first conduit and also has a deployed configuration. The second working end has a second vaned rotor and a second anchor for anchoring the second working end within the second conduit. The second working end has a delivery configuration in which it is dimensioned and shaped to be delivered to a second implantation site within the second conduit and also has a deployed configuration. The driveshaft assembly operatively interconnects the second vaned rotor with the first vaned rotor to transmit rotational movement of the second vaned rotor to the first vaned rotor. The device is motorless. When the device is implanted within the mammalian body: (a) the second vaned rotor acts as a turbine and is caused to rotate by the flow of the second fluid within the second conduit, thereby extracting kinetic energy from the second fluid, and (b) the first vaned rotor acts an impeller and is caused to rotate as a result of the rotational movement of the second vaned rotor, thereby imparting kinetic energy to the first fluid.

In some embodiments, the devices are delivered percutaneously transcatheterly. And in some such embodiments, endovascularly, as well.

In some embodiments, the first anchor includes a first wire network, and the second anchor includes a second wire network. The first wire network has a compact configuration and an expanded configuration. The first wire network is in its compact configuration when the first working end is in its delivery configuration. The first wire network is in its expanded configuration when the first working end is in its deployed configuration. The second wire network has a compact configuration and an expanded configuration. The second wire network is in its compact configuration when the second working end is in its delivery configuration. The second wire network is in its expanded configuration when the second working end is in its deployed configuration.

In some such embodiments, when the first wire network is in its expanded configuration, the first wire network surrounds the first vaned rotor and exerts a force on the first conduit sufficient to anchor the first working end in place when the device is in operation. And, when the second wire network is in its expanded configuration, the second wire network surrounds the second vaned rotor and exerts a force on the second conduit sufficient to anchor the second working end in place when the device is in operation.

In some embodiments, the first vaned rotor has a plurality of first vanes. The first vanes have a compact configuration and an expanded configuration. The first vanes are in their compact configuration when the first working end is in its delivery configuration. The first vanes are in their expanded configuration when the first working end is in its deployed configuration. And, the second vaned rotor has a plurality of second vanes. The second vanes have a compact configuration and an expanded configuration. The second vanes are in their compact configuration when the second working end is in its delivery configuration. The second vanes are in their expanded configuration when the second working end is in its deployed configuration.

In some embodiments, the first working end is overcomably biased towards its deployed configuration. Insertion of the device into a catheter overcomes the bias and causes the first working end to adopt its delivery configuration. And, the second working end is overcomably biased towards its deployed configured. Insertion of the device into the catheter overcomes the bias and causes the second working end to adopt its delivery configuration. In some such embodiments, the catheter is at least one of delivery sheath and a retrieval sheath. Similarly, in some embodiments, insertion of the device into a loader overcomes the bias and causes the first working end to adopt its delivery configuration. And, insertion of the device into the loader overcomes the bias and causes the second working end to adopt its delivery configuration. As is known in the art, the loader (once itself loaded with the device), can be used to load a delivery sheath with the device at the time of implantation.

In some embodiments, a minimum bounding right circular cylinder of the first working end when in its deployed configuration has a first diameter, a minimum bounding right circular cylinder of the second working end when in its deployed configuration has a second diameter, and the first diameter and the second diameter are unequal. In other embodiments they are equal. In the context of the present specification, a minimum bounding right circular cylinder is right circular cylinder whose axis is colinear with the longitudinal central axis of the object in question (e.g., the first working end, the second working end, etc.) and whose radius is the smallest radius possible such that the cylinder encompasses the entirety of that object. (The length of the cylinder along its longitudinal central axis is unimportant for this purpose.)

In some embodiments, the driveshaft assembly includes a driveshaft and a protective tubular covering having a lumen therein. The driveshaft is rotatably disposed with the lumen of the tubular covering. In some such embodiments, the driveshaft assembly, the driveshaft, and the covering all are flexible.

In some embodiments, the driveshaft assembly further includes gears for adjusting a ratio of rotation between the first vaned rotor and the second vaned rotor. In some embodiments, the driveshaft assembly further includes gears for reversing a direction of rotation between the first vaned rotor and the second vaned rotor. In embodiments, the driveshaft assembly further includes a clutch to operatively disconnect the first vaned rotor from the second vaned rotor.

In some embodiments, the device further comprises a first seal for sealing a first opening in a wall of the first conduit made during implantation of the device, and a second seal for sealing a second opening in a wall of the second conduit made during implantation of the device. In some such embodiments, the first seal and the second seal each radially-extend from the driveshaft assembly. In some such embodiments, the first seal and the second seal each include an expandable wire network. In some such embodiments, the first seal and the second seal each include a covered self-expanding wire network. In some such embodiments, the first seal and the second seal each assist in anchoring the device in place.

In some embodiments, the device has a first module and a second module. The first module includes the first working end and a first portion of the driveshaft assembly. The first portion of the driveshaft assembly has a first connector. The second module includes the second working end and a second portion of the driveshaft assembly. The second portion of the driveshaft assembly has a second connector. The first connector and the second connector each are structured to form an operative interconnection with the other.

In some such embodiments, the first module is dimensioned and shaped to be wholly containable within the first conduit when implanted within the mammalian body. The second module is dimensioned and shaped to be wholly containable within the second conduit when implanted within the mammalian body. The first connector and the second connector are each structured for the operative interconnection with the other without physically crossing walls of the first conduit and the second conduit. In some such embodiments, the first connector has a first magnet, the second conductor has a second magnet, and the operative interconnection between the first connector and the second connector includes a magnetic interconnection. In some such embodiments, the operative interconnection between the first connector and the second connector is solely the magnetic interconnection.

In some embodiments, the first portion of the driveshaft assembly includes a first driveshaft portion and a first protective tubular covering having a lumen therein. The first driveshaft portion is rotatably disposed within the lumen of the first protective tubular covering. The second portion of the driveshaft assembly includes a second driveshaft portion and a second protective tubular covering having a lumen therein. The second driveshaft portion is rotatably disposed within the lumen of the second protective covering. In some such embodiments, the driveshaft assembly, the first driveshaft portion, the second driveshaft portion, the first protective tubular covering and the second protective tubular covering all are flexible.

In some embodiments, the first working end is overcomably biased towards its deployed configuration. Insertion of the first module of the device into a first catheter overcomes the bias and causes the first working end to adopt its delivery configuration. The second working end is overcomably biased towards its deployed configured. Insertion of the second module of the device into a second catheter overcomes the bias and causes the second working end to adopt its delivery configuration.

In some such embodiments, the first catheter is at least one of a delivery sheath and a retrieval sheath. Similarly, the second catheter is at least one of a delivery sheath and a retrieval sheath.

Additionally, in some embodiments, insertion of the first module of the device into a first loader overcomes the bias and causes the first working end to adopt its delivery configuration. And, insertion of the second module of the device into a second loader overcomes the bias and causes the second working end to adopt its delivery configuration. As is known in the art, a loader (once itself loaded with a module of the device), can be used to load a delivery sheath with that module of the device at the time of implantation.

In some embodiments, the first working end further has a third vaned rotor and a third anchor for anchoring the first working end within the first conduit. The driveshaft assembly further operatively interconnects the second vaned rotor with the third vaned rotor to transmit the rotational movement of the second vaned rotor to the third vaned rotor. When the device is implanted within the mammalian body, the third vaned rotator acts as an additional impeller and is caused to rotate as a result of the rotational movement of the second vaned rotor, thereby imparting kinetic energy to the first fluid.

In some embodiments, the second working end further has a fourth vaned rotor and a fourth anchor for anchoring the second working end within the second conduit. The driveshaft assembly further operatively interconnects the fourth vaned rotor with the first rotor to transmit the rotational movement of the fourth vaned rotor to the first vaned rotor. When the device is implanted within the mammalian body, the fourth vaned rotator acts as an additional turbine and is caused to rotate by the flow of the second fluid within the second conduit, thereby extracting kinetic energy from the second fluid.

In some embodiments only one of the third vaned rotor and the fourth vaned rotor are present. In other embodiments, both of the third vaned rotor and the fourth vaned rotor are present. In still other embodiments, additional rotors may be present at either working end of the device.

In some embodiments, the device is elongate.

In some embodiments, each of the first vaned rotor and the second vaned rotor is an axial vaned rotor.

In some embodiments, the mammalian body is a human body. In other embodiments, the mammalian body is a non-human body (i.e., the device is intended for veterinary use).

In another aspect, implementations of the present technology provide methods of implanting a motorless intralumenal implantable fluid flow influencing device in a mammalian body. The device has a first working end. The first working end has a first vaned rotor and a first anchor for anchoring the first working end within the first conduit. The first working end has a delivery configuration in which it is dimensioned and shaped to be delivered to a first implantation site within the first conduit, and it has a deployed configuration. The device also has a second working end. The second working end has a second vaned rotor and a second anchor for anchoring the second working end within the second conduit. The second working end has a delivery configuration in which it is dimensioned and shaped to be delivered to a second implantation site within the second conduit, and it has a deployed configuration. The device also has a driveshaft assembly operatively interconnecting the second vaned rotor with the first rotor to transmit rotational movement of the second vaned rotor to the first vaned rotor.

In one implementation, the method comprises:
a) Obtaining access to a conduit system of the mammalian body of which the first conduit is a part.
b) Guiding a guidewire through the conduit system to the first implantation site within first conduit.
c) Using at least in part the guidewire to access the second conduit from the first conduit.
d) Guiding the guidewire to the second implantation site within the second conduit.
e) Railing a delivery sheath along the guidewire through the conduit system, the first conduit, and the second conduit, to the second implantation site.
f) Advancing the device with the second working end in its delivery configuration and the first working end in its delivery configuration through the delivery sheath second working end first to the second implantation site leaving the first working end of the device inside the first conduit.
g) Promoting exit of the second working end of the device from the delivery sheath at the second implantation site.
h) Causing the second working end of the device to adopt its deployed configuration anchoring the second working end of the device in place within the second conduit.
i) Promoting exit of the first working end of the device from the delivery sheath at the first implantation site.
j) Causing the first working end of the device to adopt its deployed configuration anchoring the first working end of the device in place within the first conduit.
k) Withdrawing the delivery sheath from the mammalian body.
l) Withdrawing the guidewire from the mammalian body.

In another implementation, the method comprises:
a) obtaining access to a conduit system of the mammalian body of which the second conduit is a part;
b) guiding a guidewire through the conduit system to the second implantation site within second conduit;
c) using at least in part the guidewire to access the first conduit from the second conduit;
d) guiding the guidewire to the first implantation site within the first conduit;
e) railing a delivery sheath along the guidewire through the conduit system, the second conduit, and the first conduit, to the first implantation site;
f) advancing the device with the first working end in its delivery configuration and the second working end in its delivery configuration through the delivery sheath first working end first to the first implantation site leaving the second working end of the device inside the second conduit;
g) promoting exit of the first working end of the device from the delivery sheath at the first implantation site;
h) causing the first working end of the device to adopt its deployed configuration anchoring the first working end of the device in place within the first conduit;
i) promoting exit of the second working end of the device from the delivery sheath at the second implantation site;
j) causing the second working end of the device to adopt its deployed configuration anchoring the second working end of the device in place within the second conduit;
k) withdrawing the delivery sheath from the mammalian body; and
l) withdrawing the guidewire from the mammalian body.

In some implementations, the second implantation site is downstream from an outlet of a powered ventricular assist device.

In another aspect, implementations of the present technology provide a method of implanting a mammalian body motorless intralumenal implantable fluid flow influencing device in a mammalian body. The device has a first working end. The first working end has a first vaned rotor and a first anchor for anchoring the first working end within the first conduit. The first working end has a delivery configuration in which it is dimensioned and shaped to be delivered to a first implantation site within the first conduit, and it has a deployed configuration. The device also has a second working end. The second working end has a second vaned rotor and a second anchor for anchoring the second working end within the second conduit. The second working end has a delivery configuration in which it is dimensioned and shaped to be delivered to a second implantation site within the second conduit, and it has a deployed configuration. The device also has a driveshaft assembly operatively interconnecting the second vaned rotor with the first rotor to transmit rotational movement of the second vaned rotor to the first vaned rotor. The device also has a first module. The first module includes the first working end and a first portion of the driveshaft assembly. The first portion of the driveshaft assembly has a first connector having a first magnet. The first module is dimensioned and shaped to be wholly containable within the first conduit when implanted within the mammalian body. The device also has a second module. The second module includes the second working end and a second portion of the driveshaft assembly. The second portion of the driveshaft assembly has a second connector having a second magnet. The first connector and the second connector each are structured to form an operative magnetic interconnection with the other without physically crossing walls of the first conduit and the second conduit. The second module is dimensioned and shaped to be wholly containable within the second conduit when implanted within the mammalian body.

The method comprises:
a) Obtaining first access to a conduit system of the mammalian body of which the first conduit is a part.
b) Guiding a first guidewire through the conduit system to the first implantation site within first conduit.
c) Railing a first delivery sheath along the first guidewire through the conduit system and the first conduit to the first implantation site.
d) Advancing the first module of the device with the first working end in its delivery configuration through the first delivery sheath the first portion of the driveshaft assembly first to the first implantation site.
e) Promoting exit of the first module of the device from the first delivery sheath at the first implantation site.
f) Causing the first working end of the device to adopt its deployed configuration anchoring the first working end of the device in place within the first conduit.
g) Withdrawing the first delivery sheath from the mammalian body.
h) Withdrawing the first guidewire from the mammalian body.
i) Obtaining second access to a conduit system of the mammalian body of which the second conduit is a part.
j) Guiding a second guidewire through the conduit system to the second implantation site within second conduit.
k) Railing a second delivery sheath along the second guidewire through the conduit system and the second conduit to the second implantation site.
l) Advancing the second module of the device with the second working end in its delivery configuration through the second delivery sheath the second portion of the driveshaft assembly first to the second implantation site.
m) Promoting exit of the second module of the device from the second delivery sheath at the second implantation site such that the first connector and the second connector form the operative magnetic interconnection with each other without physically crossing walls of the first conduit and the second conduit.
n) Causing the second working end of the device to adopt its deployed configuration anchoring the second working end of the device in place within the second conduit.
o) Withdrawing the second delivery sheath from the mammalian body,
p) Withdrawing the second guidewire from the mammalian body.

In some implementations, the second implantation site is downstream from an outlet of a powered ventricular assist device.

It should be understood that in some implementations the conduit system of the mammalian body of which the first conduit is a part and the conduit system of the mammalian body of which the second conduit is a part, may, in fact, be different parts of the same conduit system. Alternatively, in other implementations, they may, in fact, be different conduit systems.

Finally, in some implementations, a) to h) are completed prior to i) to p). In other implementations, i) to p) are completed prior to a) to h). In still other implementations, a) to h) are partially or fully completed at the same time as i) to p).

### General

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “first unit” and “third unit” is not intended to imply any particular type, hierarchy or ranking (for example) of/between the units. Nor is their use (by itself) intended imply that any “second unit” must necessarily exist in any given situation.

In the context of the present specification, the word “embodiment(s)” is generally used when referring to physical realizations of the present technology and the word “implementations” is generally used when referring to methods that are encompassed within the present technology (which generally involve also physical realizations of the present technology). The use of these different terms is not intended to be limiting of or definitive of the scope of the present technology. These different terms have simply been used to allow the reader to better situate themselves when reading the present lengthy specification.

Embodiments and implementations of the present technology each have at least one of the above-mentioned objects and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of embodiments and/or implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following detailed description of some embodiments and implementations, which is to be used in conjunction with the accompanying drawings, where:

FIG. 1 is a prior art device as shown in FIG. 1 of the WO '946 Publication.

FIG. 2 is a perspective view of a first embodiment of a device of the present technology taken from the first working end thereof, shown with the first working end and the second working end being in their deployed configurations.

FIG. 3 is an isometric view of the device of FIG. 2, shown with the first working end and the second working end being in their deployed configurations.

FIG. 4 is a close-up isometric view of the first working end of the device of FIG. 2, shown in its deployed configuration; in this embodiment the second working end is a mirror image thereof.

FIG. 5 is a close-up side elevation view of the first working end of the device of FIG. 2, shown in its deployed configuration; in this embodiment second working end is a mirror image thereof.

FIG. 6 is an isometric view of the device of FIG. 2, similar to that of FIG. 3, but having the protective covering of the driveshaft assembly removed.

FIG. 7 is an exploded isometric view of the device of FIG. 2, similar to that of FIG. 3.

FIG. 8 is a side elevation view of the device of FIG. 2, shown with the first working end anchor and the second working end anchor removed.

FIG. 9 is an isometric view of the device of FIG. 2, shown with the first working end anchor and the second working end anchor removed, similar to FIG. 8.

FIG. 10 is a side cross-section view of the device of FIG. 2, similar to FIG. 8, shown with the first working end anchor and the second working end anchor removed.

FIG. 11 is a side cross-section view of the device of FIG. 2, similar to FIG. 10, shown with the first working end anchor and the second working end anchor in their deployed configurations.

FIG. 12 is a side elevation view of the device of FIG. 2, shown in a catheter, with the first working end and the second working end in their delivery configurations.

FIG. 13 is a side cross-section view of the device of FIG. 2 similar to FIG. 12, shown in a catheter, with the first working end and the second working end in their delivery configurations.

FIG. 14 is an isometric view of a second embodiment of a device of the present technology, shown with the first working end and the second working end being in their deployed configurations.

FIG. 15 is an isometric view of a third embodiment of a device of the present technology, showing both the first module and the second module of the device, shown with the first working end (of the first module) and the second working end (of the second module) being in their deployed configurations.

FIG. 16 is an isometric view of the third embodiment of a device of the present technology, showing both the first module and the second module of the device, shown with the protective coverings of the driveshaft assembly removed, and shown with the first working end (of the first module) and the second working end (of the second module) being in their deployed configurations.

FIG. 17 is a side elevation view of a fourth embodiment of a device of the present technology, shown with the first working end and the second working end being in their deployed configurations.

FIG. 18 is a side elevation view of a fifth embodiment of a device of the present technology, shown with the first working end and the second working end being in their deployed configurations.

FIG. 19 is a side elevation view of a sixth embodiment of a device of the present technology, shown with the first working end and the second working end being in their deployed configurations.

FIG. 20 is a first schematic anatomical view of the first embodiment of a device of the present technology with the first working end being disposed in the pulmonary artery and the second working end being disposed in the aorta.

FIG. 21 is a second schematic anatomical view of the first embodiment of a device of the present technology with the first working end being disposed in the pulmonary artery and the second working end being disposed in the aorta.

FIG. 22 is a third schematic anatomical view of the first embodiment of a device of the present technology with the first working end being disposed in the pulmonary artery and the second working end being disposed in the ascending aorta.

FIG. 23 is a first schematic anatomical view of the first embodiment of a device of the present technology with the first working end being disposed in the inferior vena cava and the second working end being disposed in the abdominal aorta.

FIG. 24 is a second schematic anatomical view of the first embodiment of a device of the present technology with the first working end being disposed in the inferior vena cava and the second working end being disposed in the abdominal aorta.

FIG. 25 is an isometric view of a seventh embodiment of a device of the present technology, shown with the first working end, the second working end, and a sealing unit each being in their deployed configurations.

FIG. 26 is an isometric view of the sealing unit of the seventh embodiment of the device shown in FIG. 25, shown in its deployed configuration.

FIG. 27 is an isometric view of an eighth embodiment of a device of the present technology, shown with a first sealing unit and a second sealing unit being in their deployed configurations.

FIG. 28 is a side elevation view of the eighth embodiment of the device shown in FIG. 27, shown with the first sealing unit and a second sealing unit being in their deployed configurations.

FIG. 29 is an isometric view of the first sealing unit of the eighth embodiment of the device shown in FIG. 27; in this embodiment the second sealing unit of the device is identical to the first sealing unit.

FIG. 30 is a side elevation view of the first sealing unit shown in FIG. 29 of the eighth embodiment of the device shown in FIG. 27.

FIG. 31 is an isometric view of a ninth embodiment of a device of the present technology.

FIG. 32 is another isometric view of the ninth embodiment of a device of the present technology.

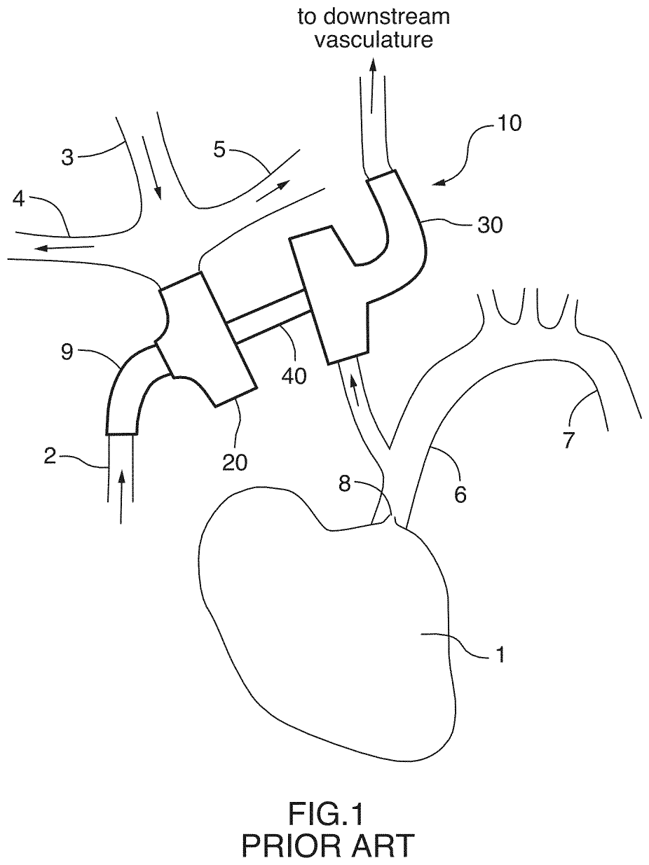

### DETAILED DESCRIPTION OF SOME EMBODIMENTS AND IMPLEMENTATIONS

### Introduction

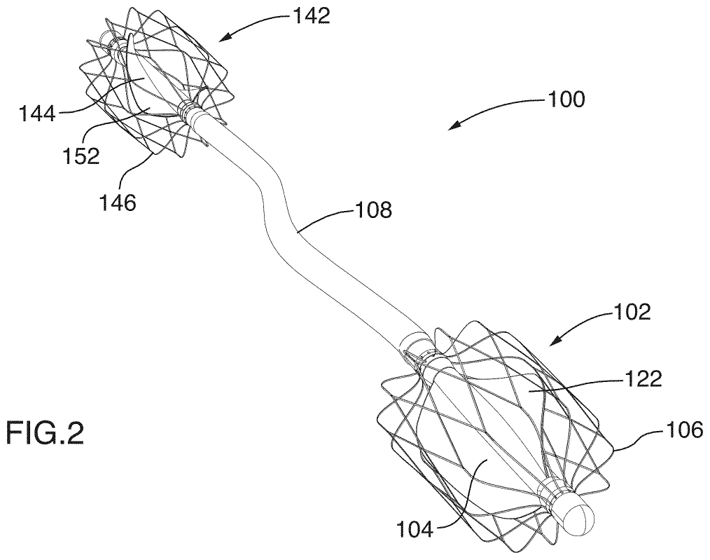

Referring to FIG. 2, there is shown a mammalian body implantable fluid flow influencing device 100, being a first embodiment of the present technology. It is to be expressly understood that the device 100 is merely one embodiment, amongst many, of the present technology. Other embodiments are also described hereinbelow. Thus, the description thereof that follows is intended to be only a description of illustrative examples of the present technology. This description is not intended to define the scope or set forth the bounds of the present technology. In some cases, what are believed to be helpful examples of modifications to the device 100 and/or other embodiments may also be set forth hereinbelow. This is done merely as an aid to understanding, and, again, not to define the scope or set forth the bounds of the present technology. These modifications are not an exhaustive list, and, as a skilled addressee would understand, other modifications are likely possible. Further, where this has not been done (i.e., where no examples of modifications have been set forth), it should not be interpreted that no modifications are possible and/or that what is described is the sole manner of implementing that element or feature of the present technology. As a skilled addressee would understand, this is likely not the case. In addition, it is to be understood that the device 100 may provide in certain instances a simple embodiment of the present technology, and that where such is the case it has been presented in this manner as an aid to understanding. As a skilled addressee would understand, various embodiments of the present technology are of a greater complexity.

### General Description

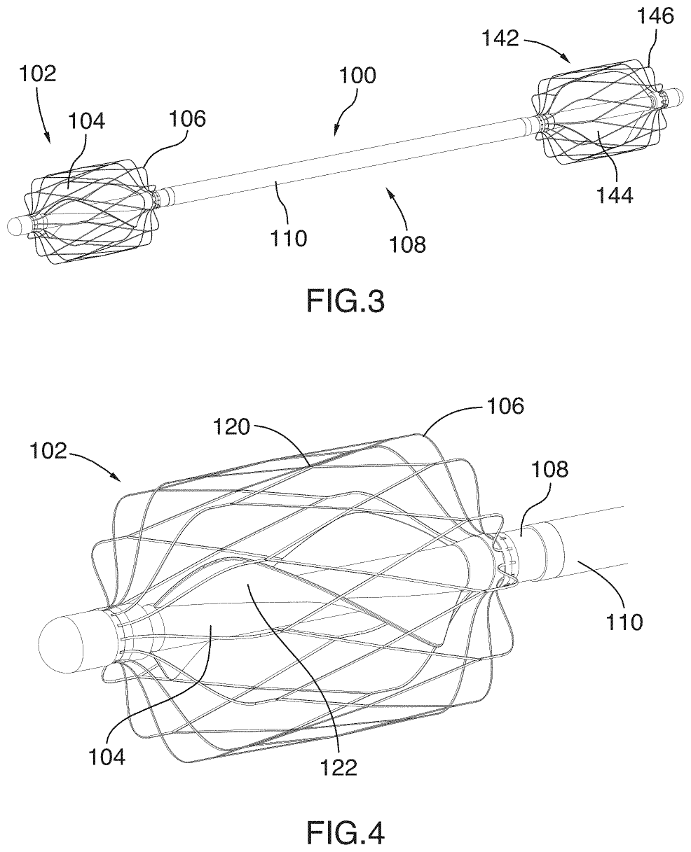

The embodiment of the device 100 shown in FIGS. 2 and 3, has a first working end 102 and a second working end 142. The first and second working ends 102, 142 are connected together by a flexible driveshaft assembly 108. First working end 102 has a first vaned rotor 104, being a first vaned axial impeller. First vaned rotor 104 has first vanes 122. First working end 102 also has a first anchor 106, being first wire network. Second working end 142 has a second vaned rotor 144, being a second vaned axial turbine. Second vaned rotor 144 has second vanes 152. Second working end 122 also has a second anchor 146, being second wire network. In this embodiment (for purposes of facilitating understanding) first and second working ends 102, 142 are identical. In other embodiments this is not case.

The device 100 has no motor.

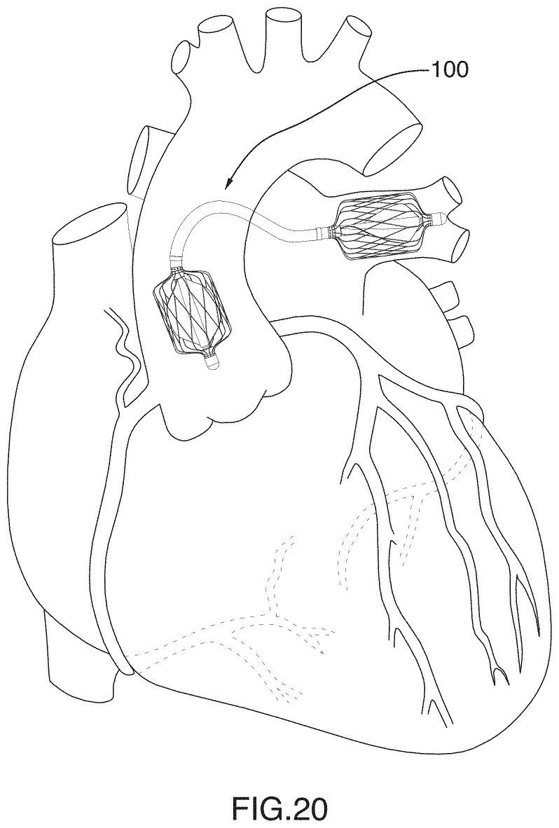

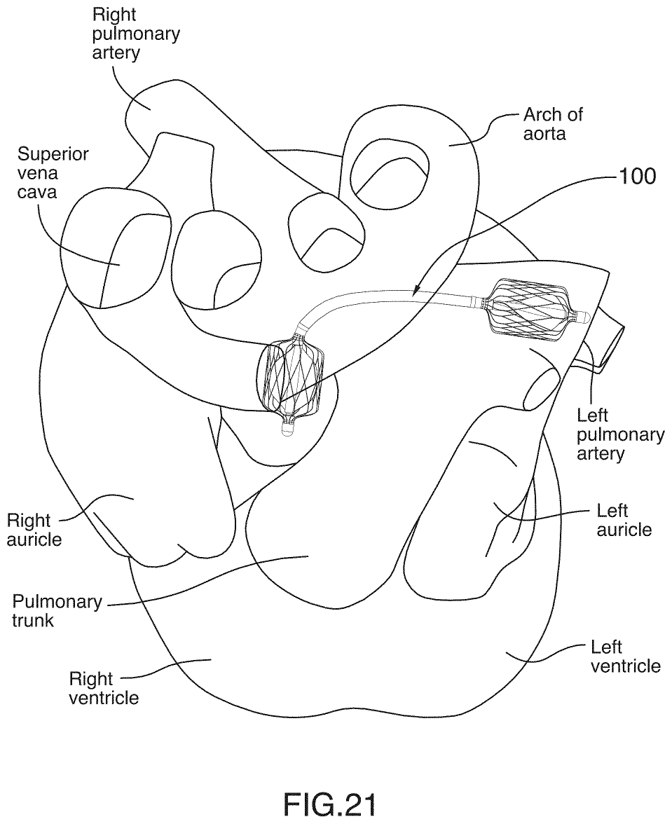

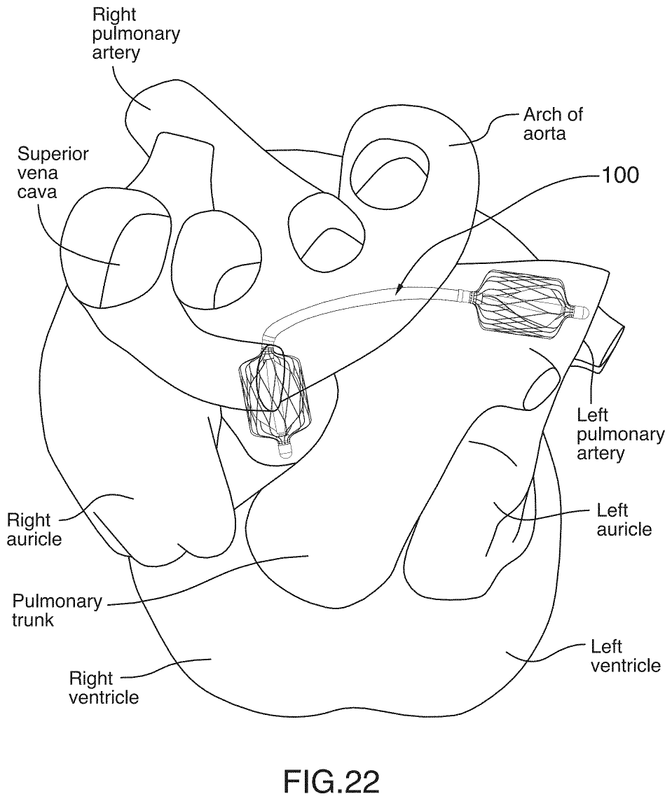

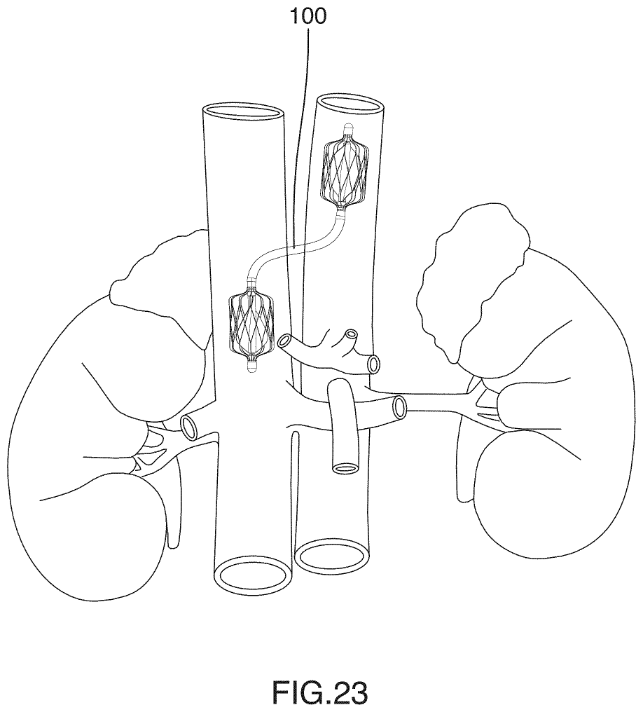

Referring to FIGS. 20-22, as a first example, the first working end 102 of the device 100 is implanted within left pulmonary artery of a human and the second working end 142 of the device 100 is implanted within an ascending aorta of the human. In this use case, the second vaned rotor 144 of the device 100 is caused to rotate as a result of blood flowing through the ascending aorta. The second vaned rotor 144 thus acts as a turbine and extracts kinetic energy from the flowing blood. The rotational movement of the second vaned rotor 144 is transmitted to the driveshaft assembly 108 and then to the first vaned rotor 102. The first vaned rotor 102 is thus caused to rotate and acts as an impeller imparting kinetic energy to the blood flowing through the left pulmonary artery.

Although only shown schematically in the drawings, the first anchor 106 is an expanded configuration with the wires 120 of the first wire network exerting sufficient force on the walls of the left pulmonary artery to anchor the first working end 102 in place during operation of the device 100. Similarly, the second anchor 146 is an expanded configuration with the wires 150 of the second wire network exerting sufficient force on the walls of the ascending aorta to anchor the second working end 142 in place during operation of the device 100.

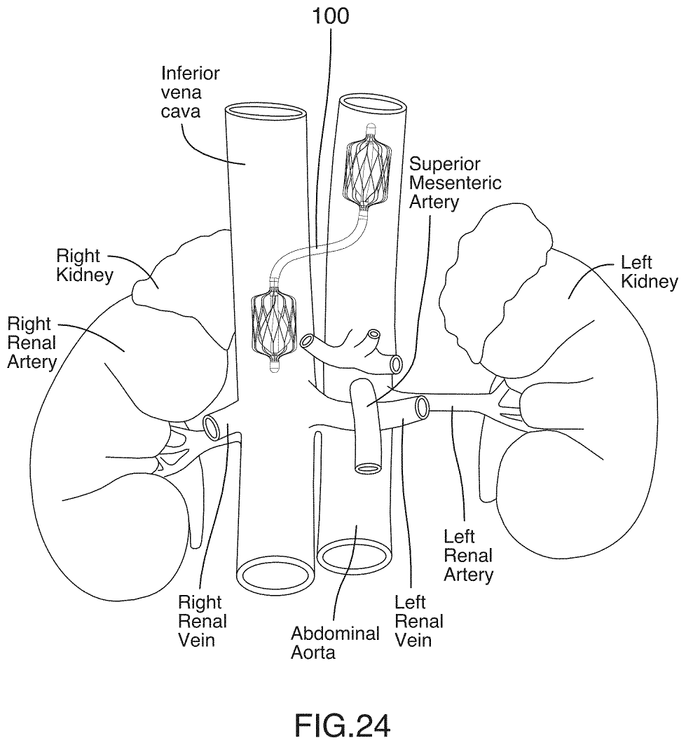

Referring to FIGS. 23-24, as a second example, the first working end 102 of the device 100 is implanted within the inferior vena cava of a human and the second working end 142 of the device 100 is implanted within the abdominal aorta of the human. In this use case, the second vaned rotor 144 of the device 100 is caused to rotate as a result of blood flowing through the abdominal aorta. The second vaned rotor 144 thus acts as a turbine and extracts kinetic energy from the flowing blood. The rotational movement of the second vaned rotor 144 is transmitted to the driveshaft assembly 108 and then to the first vaned rotor 102. The first vaned rotor 102 is thus caused to rotate and acts as an impeller imparting kinetic energy to the blood flowing through the inferior vena cava.

Although only shown schematically in the drawings, the first anchor 106 is an expanded configuration with the wires 120 of the first wire network exerting sufficient force on the walls of the inferior vena cava to anchor the first working end 102 in place during operation of the device 100. Similarly, the second anchor 146 is an expanded configuration with the wires 150 of the second wire network exerting sufficient force on the walls of the abdominal aorta to anchor the second working end 142 in place during operation of the device 100.

Depending on many factors, including but not limited to, the state of the health of the patient, the particulars of the assistance the device is to render, the characteristics of the device, and the particulars of the implantation, the device 100 may be implanted on its own (as is shown in the examples in FIG. 20-24) or may be implanted downstream of a powered VAD (not shown in the drawings). One benefits of employing the device 100 in combination with a powered VAD is that the characteristics of the VAD, those of the device 100 and those of both of their operations can take the combined presence of both into account. Thus, helping to ensure that sufficient energy may be imparted by the VAD to the blood flowing in a first bodily conduit such that sufficient energy can be removed from that flowing blood by the device 100 and transferred to the fluid flowing in the second bodily conduit in which the other end of the device 100 is implanted, while still leaving sufficient energy in the blood flowing in the first conduit as is necessary.

Thus, for example, in the case where an LVAD is implanted into a patient, at a high level, the electrical energy supplied to the LVAD (e.g., from a battery or some other power source) is converted by the LVAD's pumping unit into the kinetic energy of the increased blood flow flowing out of the pump. Downstream of the pump outlet, some of this kinetic energy can be extracted from the flowing blood by the device and used to do other work elsewhere in the body without having its own motor or power source.

### First Vaned Rotor (Impeller) & Second Vaned Rotor (Turbine)

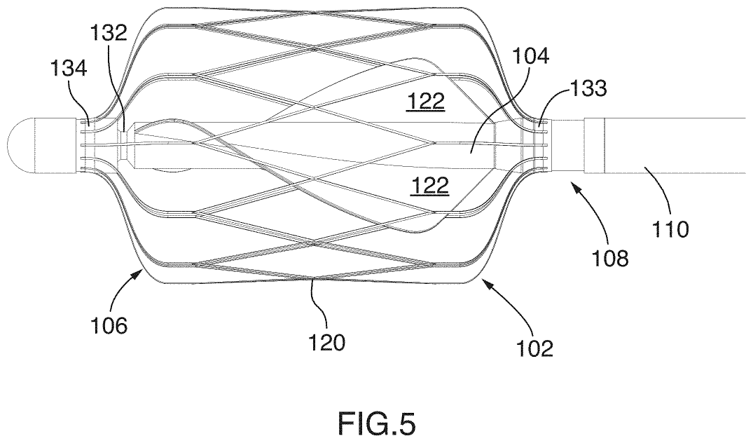

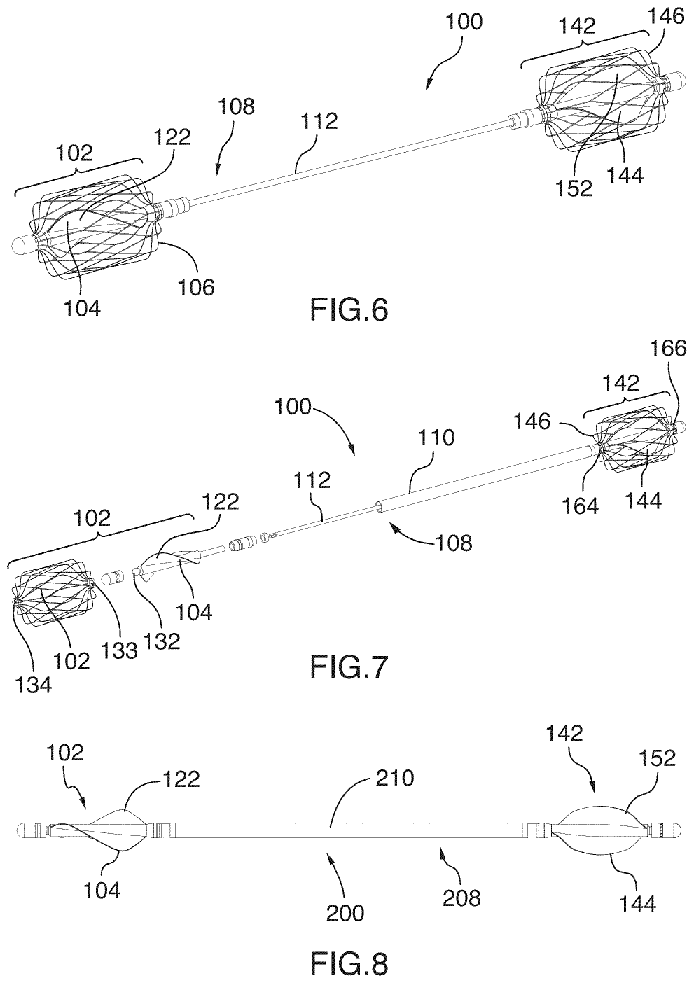

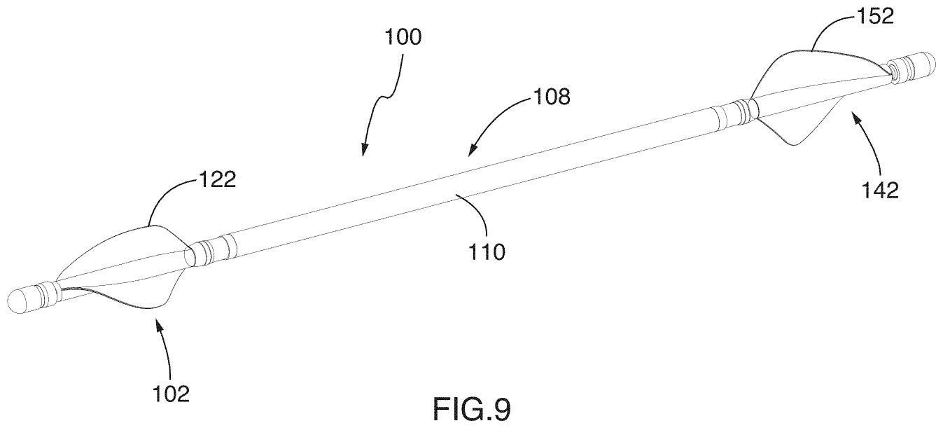

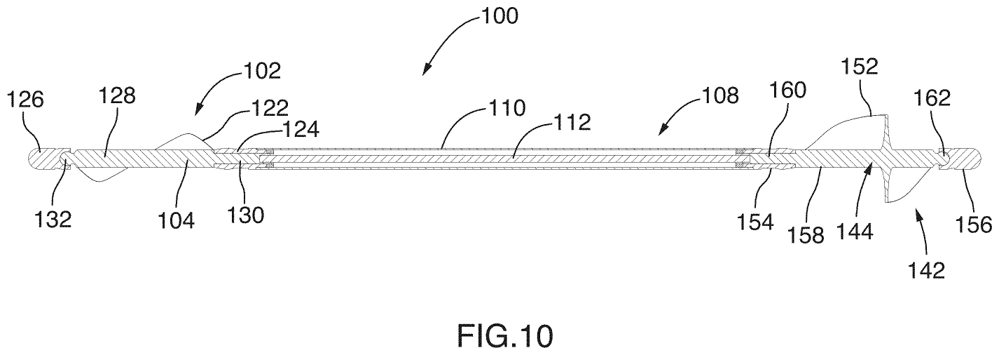

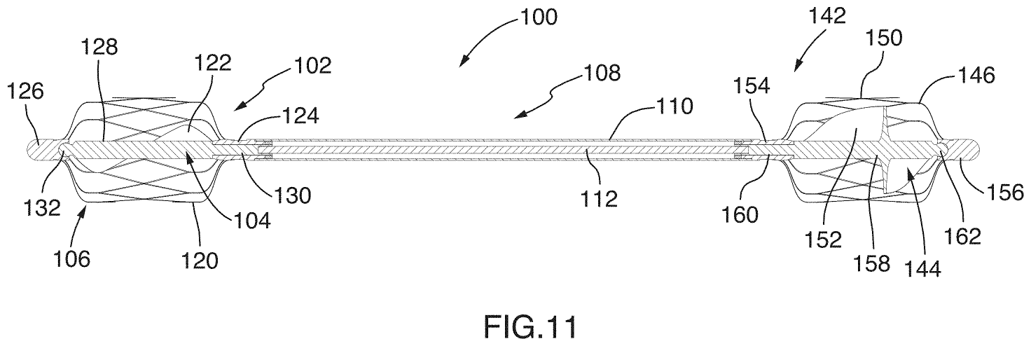

Referring to FIGS. 3-11, the first vaned rotor 104 has a hub 128 and vanes 122 projecting therefrom. Extending from the hub 128 is a shaft 130 and a spindle 132. The shaft 130 is connected to the driveshaft 112 (which is part of the driveshaft assembly 108), such that when the driveshaft 112 rotates, the shaft 130 rotates, rotating the hub 128 and the vanes 122, operating the first vaned rotor 104 (as an impeller). The shaft 130 is surrounded by and maintained in place by a first connector 124. The first connector 124 has bearings therein that allow the shaft 130 to rotate freely within the first connector 124. The first connector 124 itself does not rotate. At the opposite end of the hub 128 is the spindle 132. The spindle 132 is rotatably seated within the first end cap 126 such that the spindle 132 can rotate freely while the end cap 126 does not.

In this embodiment, the first vaned rotor 104 is made of titanium, but in other embodiments the first vaned rotor 104 may be made of any other appropriate medical grade material(s). In this embodiment the first vanes 122 are not expandable (as the skilled addressee would understand, expandable vanes are known in the art). In other embodiments, the first vanes 122 may be expandable. In either case, the maximum cross-sectional diameter of the first vaned rotor 104 is typically between 1 mm (3 Fr) and 12 mm (36 Fr) (when expanded in the case of expandable vanes), depending on the patient, the implantation site, etc, as the skilled address would understand. The typical length of the hub 128 is between 3 mm and 50 mm, again depending on the patient, the implantation site, etc., as the skilled address would understand. Finally, the present technology does not require any specific design for the vanes 122 of the first vaned rotor 104. As a skilled addressee would understand appropriate rotor vane design for a particular circumstance is known in the art.

As was mentioned hereinabove, in this embodiment, the structure of the second working end 142 is the same as that of the first working end 102. Thus, again referring to FIGS. 3-11, the second vaned rotor 144 has a hub 158 and vanes 152 projecting therefrom. Extending from the hub 158 is a shaft 160 and a spindle 162. The shaft 160 is connected to the driveshaft 112 (which is part of the driveshaft assembly 108), such that when the second vaned rotor 144 operates (as a turbine), the flowing fluid acts on the vanes 142 to cause the hub 158 to rotate, rotating the driveshaft 112. The shaft 160 is surrounded by and maintained in place by a second connector 154. The second connector 154 has bearings therein that allow the shaft 152 to rotate freely within the second connector 154. The second connector 154 itself does not rotate. At the opposite end of the 158 is the spindle 162. The spindle 162 is rotatably seated within the second end cap 156 such that the spindle 162 can rotate freely while the end cap 156 does not.

In this embodiment, the second vaned rotor 144 is made of titanium, but in other embodiments the second vaned rotor 144 may be made of any other appropriate medical grade material(s). In this embodiment, the second vanes 152 are not expandable. In other embodiments, the second vanes 152 may be expandable. In either case, the maximum cross-sectional diameter of the second vaned rotor 144 is typically between 1 mm (3 Fr) and 12 mm (36 Fr) (when expanded in the case of expandable vanes), depending on the patient, the implantation site, etc., as the skilled address would understand. The typical length of the hub 158 is between 3 and 50 mm, again depending on the patient, the implantation site, etc., as the skilled address would understand. Finally, the present technology does not require any specific design for the vanes of the second vaned rotor 144. As a skilled addressee would understand, appropriate rotor vane design for a particular circumstance is known in the art.

### First Anchor (Wire Network) & Second Anchor (Wire Network)

As can best be seen in FIGS. 4, 5 and 11, the first working end 102 has a first anchor 106. In this embodiment, the first anchor 106 is a wire network that surrounds the first vaned rotor 104 (the impeller). Wires 120 of the first wire network are attached at one end to the outer portion of the first connector 124 via their connection to a metal band 133 encircling a portion of the outer surface of the first connector 124. Further, at the other end, wires 120 of the wire network are attached to the first end cap 126 via their connection to a metal band 134 encircling a portion of the outer surface of the first end cap 126. Thus, the first anchor 106 (the first wire network) is attached at both ends to portions of the device 100 that are independent from the rotation of the first vaned rotor 104. This allows the first anchor 106 to anchor the first working end 102 of the device 100 within a bodily conduit while not hindering rotation of the first vaned rotor 104.

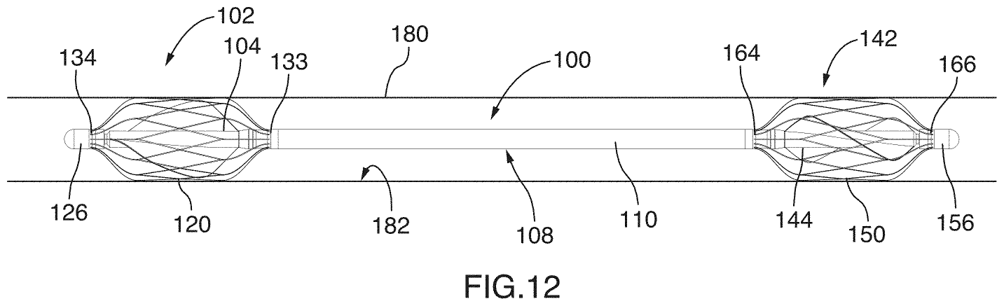

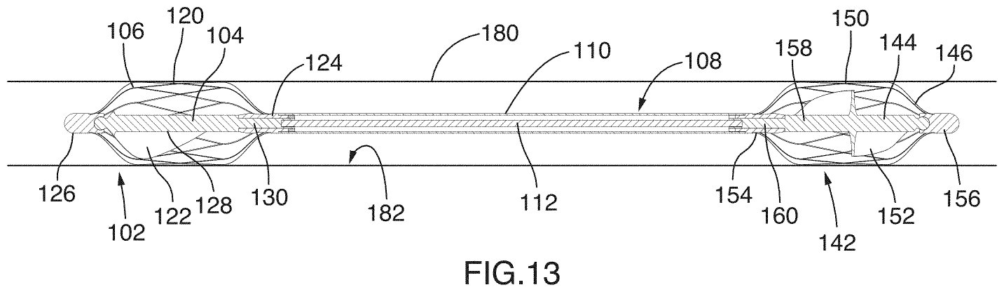

The first anchor 106 has an expanded configuration (shown in FIGS. 2-11) and a compact configuration (shown in FIGS. 12 and 13). In the expanded configuration, the first anchor 106 is of such a size (e.g., diameter or effective diameter (in the context of the present specification, an effective diameter is the diameter of minimum bounding right circular cylinder (which is itself defined hereinebelow))) that it exerts sufficient force on the wall of a bodily conduit into which the first working end 102 has been implanted to anchor the first working end 102 in place. Further, in the expanded configuration the first anchor 106 is sufficiently distant from the first vaned rotor 104 so as not to come into contact with the vanes 122 of the first vaned rotor 104 when the rotor 104 is rotating (in operation). Thus, in addition to its anchoring function, in this embodiment, the first anchor 106 also serves the function of protecting the walls of the conduit into which the first working end 102 has been implanted from being damaged by the vanes 122 of the first vaned rotor 104 when the rotor 104 rotates.

In this embodiment, the first anchor 106 (the first wire network) is biased towards its expanded configuration, but that bias can be overcome via insertion into the lumen 184 of an appropriately sized catheter (e.g., delivery sheath, retrieval sheath) 180 (or a loader) which has a smaller internal diameter than the diameter or the effective diameter of the first anchor 106, e.g., see FIGS. 12 and 13. As the first anchor 106 is inserted into the lumen 184, contact of the wires 120 of the first anchor 106 with the walls 182 of the catheter 180 (or loader—as the case may be) overcomes the bias and forces the first anchor 106 to compact in on itself. This brings the wires 120 closer in towards the first vaned rotor 104 (the impeller) (i.e., in a direction towards the longitudinal central axis of the first vaned rotor 104) as the first anchor 106 adopts its compact configuration. Depending on the characteristics of the device 100 and the catheter 180 (or loader), e.g., the size of the lumen of the catheter 180 (or loader), the size and design of the first wire network, etc., the wires 120 may contact the vanes 122 and/or the hub 128 as the first anchor 106 adopts its compact configuration. This situation is generally tolerable when the first anchor 106 is its compact configuration as the first vaned rotor 104 will not be operated when the first anchor 106 is in that configuration. As the first anchor 106 is biased towards its expanded configuration, when the first anchor 106 exits the catheter 180 at the first implantation site within the first conduit, the first anchor 106 adopts its expanded configuration (which was described above). As the skilled addressee would understand, when the device 100 is in a loader, the loader is used to load the device into, typically, a delivery sheath 180. In such situations, the first anchor 106 would remain in its compact configuration when transferred from the loader to the delivery sheath 180.

In this embodiment, the first anchor 106 (first wire network) is made of nitinol, a medical grade shape memory alloy. It is the “shape memory” of the nitinol that causes the bias of the first anchor 106 towards its expanded configuration. But, as the skilled addressee would understand, nitinol is deformable from its “remembered shape” in use, without “forgetting” that shape. Thus, it returns to that shape when the forces having caused its deformation are removed. In other embodiments, the first anchor 106 can be made of any other suitable medical grade material or combination of material(s) having the appropriate design characteristics.

As can best be seen in FIGS. 4, 5 and 11, the second working end 142 has a second anchor 146. In this embodiment, the second anchor 146 is a wire network that surrounds the second vaned rotor 144. Wires 150 of the wire network are attached at one end to the outer portion of the second connector 154 via their connection to a metal band 164 encircling a portion of the outer surface of the second connector 154. Further, at the other end, wires 150 of the wire network are attached to the second end cap 156 via their connection to a metal band 166 encircling a portion of the outer surface of the second end cap 156. Thus, the second anchor 146 is attached at both ends to portions of the device 100 that are independent from the rotation of the second vaned rotor 144. This allows the second anchor 146 to anchor the second working end 142 of the device 100 within a bodily conduit while not hindering rotation of the second vaned rotor 144.

The second anchor 146 (second wire network) has an expanded configuration (shown in FIGS. 2-11) and a compact configuration (shown in FIGS. 12 and 13). In the expanded configuration, the second anchor 146 is of such a size (e.g., diameter or effective diameter) that it exerts sufficient force on the wall of a bodily conduit into which the second working end 142 has been implanted to anchor the second working end 142 in place. Further, in the expanded configuration the second anchor 146 is sufficiently distant from the second vaned rotor 144 so as not to come into contact with the vanes 152 of the second vaned rotor 144 when the rotor 144 is rotating (in operation). Thus, in addition to its anchoring function, in this embodiment, the second anchor also serves the function of protecting the walls of the conduit into which the second working end 142 has been implanted from being damaged by the vanes 152 of the second vaned rotor 144 when the rotor 144 rotates.

In this embodiment, the second anchor 146 (second wire network) is biased towards its expanded configuration, but that bias can be overcome via insertion into the lumen 184 of a catheter (e.g., delivery sheath, retrieval sheath) 180 (or a loader) which has a smaller internal diameter than the diameter or the effective diameter of the second anchor 146, see FIGS. 12 and 13. As the second anchor 146 is inserted into the lumen 184, contact of the wires 150 of the second anchor 146 with the walls 182 of the catheter 180 or loader (as the case may be) overcomes the bias and forces the second anchor 146 to compact in on itself. This brings the wires 150 closer in towards the second vaned rotor 144 (the turbine) (i.e., in a direction towards the longitudinal central axis of the second vaned rotor 144) as the second anchor 146 adopts its compact configuration. Depending on the characteristics of the device 100 and the catheter 108 (or loader), e.g., the size of the lumen of the catheter 180 (or loader), the size and design of the second wire network, etc., the wires 150 may contact the vanes 152 and/or the hub 158 as the second anchor 146 adopts (and is in) its compact configuration. This situation is generally tolerable when the second anchor 146 is its compact configuration as the second vaned rotor 144 will not be operated when the second anchor 146 is in that configuration. As the second anchor 146 is biased towards its expanded configuration, when the second anchor 146 exits the catheter 180 at the second implantation site within the second conduit, the second anchor 146 adopts its expanded configuration (which is described above.) As the skilled addressee would understand, when the device 100 is in a loader, the loader is used to load the device into, typically, a delivery sheath 180. In such situations, the second anchor 146 would remain in its compact configuration when transferred from the loader to the delivery sheath 180.

In this embodiment, the second anchor 146 (second wire network) is made of nitinol, a medical grade shape memory alloy. In other embodiments, the second anchor 146 can be made of any other suitable medical grade material or combination of material(s) having the appropriate design characteristics.

### First Working End & Second Work End Configurations

As the skilled addressee would understand, the size of catheters for use in human beings is measured according to the French scale (Fr). Such catheters commonly vary in outer diameter between 3 Fr (1 mm) and 36 Fr (12 mm). (The Fr scale may be converted to millimetres by dividing the Fr by 3.). So, for example, if it were determined that a 6 Fr catheter were to be used in a particular procedure, any components to be delivered through that catheter must be selected such that their dimensions and shapes will permit them to be delivered through a catheter of 6 Fr. Thus, in the present context, the device 100 must meet such a limitation.

Depending on the particular patient and the particular implantation sites of the first working end 102 and the second working end 142 of the device 100, the size of the catheter 180 required may vary. For example, were the device 100 to be implanted within a patient suffering from coronary artery disease with heart failure, their peripheral vasculature through which the catheter 180 must pass may be partially blocked by peripheral artery disease and thus have reduced cross-sectional area as compared with that of a person not suffering from that disease. The surgeon would thus have to select the appropriately sized catheter 180 and device 100 such that the catheter 180 can pass through the minimum available cross-section of the blood vessels, to the implantation sites, and the device 100 can be delivered (e.g., can themselves pass through) via the catheter 180 to the implantation sites as appropriate.

The first working end 102 and the second working end 142 of the device 100 each have a delivery configuration and a deployed configuration. When in its delivery configuration (e.g., in the catheter 180), the first working end 102 of the device 100 is dimensioned and shaped to be delivered to the first implantation site within the first conduit. Thus, for example, in this embodiment, assuming a 15 Fr (5 mm) catheter 180 were appropriate, the first working end 102 could have an effective diameter of no larger than 5 mm when in its delivery configuration; meaning that both the first vaned rotor 104 and the first anchor 106 in its compact configuration could have an effective diameter of no larger than 5 mm themselves. Similarly, continuing with the same example, the second working end 142 could have an effective diameter of no larger than 5 mm when in its delivery configuration; meaning that both the second vaned rotor 144 and the second anchor 146 in its compact configuration could have an effective diameter of no larger than 5 mm themselves as well.

In a similar example, in another embodiment, where the first vaned rotor and the second vaned rotor were themselves expandable and therefore had their own compact and expanded configurations, neither the first vaned rotor nor the second vaned rotor could have an effective diameter of no larger than 5 mm when in their compact configurations.

It should be understood, however, that the maximum effective diameter of the first working end 102 and the second working end 142 in their delivery configurations (and thus the maximum effective diameter of the first anchor 106, the second anchor 146, the first vaned rotor 104, and the second vaned rotor 144 in their compact configurations (in embodiments where they have them)) does not necessarily dictate the size and shape of the first working end 102 and the second working end 142 in their deployed configurations (and thus the maximum effective diameter of the first anchor 106, the second anchor 146, the first vaned rotor 104, and the second vaned rotor 144 in their expanded configurations (in embodiments where they have them)). Thus, while the maximum effective diameter of the first working end 102 and the second working end 142 may be the same for both of them when they are in their delivery configurations, that is not necessarily the case when they are in their deployed configurations. As the first working end 102 is to be implanted at the first implantation site within the first conduit of a particular patient, in its deployed configuration the first working end 102 (and thus the first vaned rotor 104 and the first anchor 106 in their expanded configurations, if such exist in that embodiment) needs to be selected in accordance with the relevant characteristics of the first conduit at the first implantation site in that patient. Similarly, as the second working end 142 is to be implanted at the second implantation site within the second conduit in that patient, in its deployed configuration the second working end 142 (and thus the second vaned rotor 144 and the second anchor 146 in their expanded configurations, if such exist in that embodiment) needs to be selected in accordance the relevant characteristics of the second conduit at the second implantation site in that patient. Thus, in many embodiments, the first working end 102 and the second working end 142 will be differently configured in their deployed configurations.

### Drive Shaft Assembly

In this embodiment of the device 100, as can be best seen in FIGS. 7, 10, 11, and 13, a driveshaft assembly 108 operatively interconnects the first vaned rotor 104 (impeller) with the second vaned rotor 144 (turbine) to transmit rotational movement of the second vaned rotor 144 to the first vaned rotor 104. The driveshaft assembly 108 (in this embodiment) includes driveshaft 112, protective hollow tubular covering 110, first connector 124 (at the first working end 102) and second connector 154 (at the second working end 142).

At the first working end 102 of the device 100, one end of the protective hollow tubular covering 110 is connected to the outer portion of the first connector 124 so as to be independent from rotational movement of the driveshaft 112 (described below). At the second working end 142 of the device 100, the other end of the protective hollow tubular covering is connected to the outer portion of the second connector 154 so as also to be independent from rotational movement of the driveshaft 112.

At the first working end 102 of the device 100, within a lumen of the first connector 124, one end of the driveshaft 112 is connected to the shaft 130 of the first vaned rotor 104. Both the shaft 130 and the end of the driveshaft 112 are rotatably supported within the lumen of the first connector 124 (e.g., by appropriate bearings). Thus, the shaft 130 and the driveshaft 112 can rotate freely without rotating the first connector 124 itself. At the second working end 142 of the device 100, within a lumen of the second connector 154, the other end of the driveshaft 112 is connected to the shaft 160 of the second vaned rotor 144. Both the shaft 160 and the end of the driveshaft 112 are rotatably supported within the lumen of the second connector 154 (e.g., by appropriate bearings). Thus, the shaft 160 and the driveshaft 112 can rotate freely without rotating the second connector 154 itself.

The driveshaft 112 and the lumen of the of the protective hollow tubular covering 110 are sized such that the covering 110 does not interfere with the rotation of the driveshaft 112 during operation of the device. The protective hollow tubular covering 110 covers the entirety of the driveshaft 112 so that no part of the driveshaft is exposed to any part of the patient's body, thus reducing the risk that the driveshaft 112 causes damage within the patient's body. In this embodiment, both the driveshaft 112 and the protective tubular hollow covering 110 are flexible, such that they may conform to a non-linear path between the first working end and the second working end of the device without causing harm to the patient (e.g., see FIGS. 20-24).

The driveshaft assembly 108 is made of conventional materials and is of a design (similar to that of conventional driveshafts of, e.g., intralumenal VADs).

In the embodiment shown in FIGS. 2 to 13, the driveshaft assembly 108 contains no gears or other structures that alter the rotational movement between the second vaned rotor 144 (the turbine) and the first vaned rotor 104 (the impeller). Thus, the second vaned rotor 144 and the first vaned rotor 104 rotate in a 1:1 ratio in this embodiment, with rotational movement of the second vaned rotor 144 (the turbine) always being transmitted by the driveshaft 112 to the first vaned rotor 104 (the impeller) when the second vaned rotor 144 rotates.

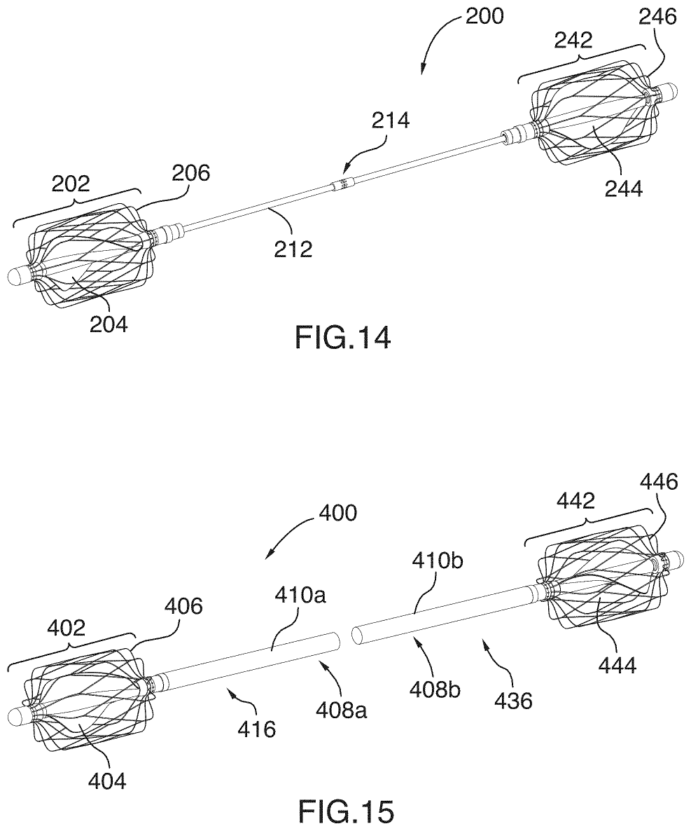

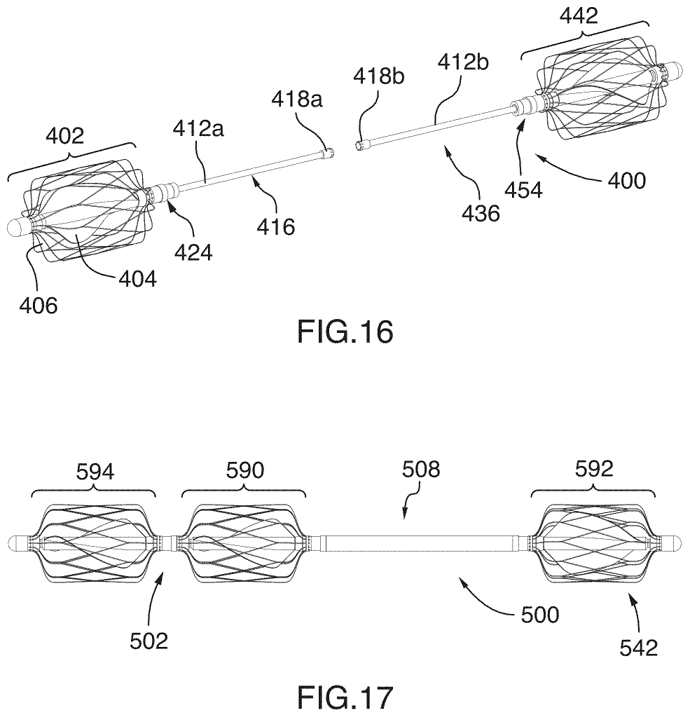

This is not the case, however, in other embodiments. For example, FIG. 14 shows second embodiment of the present technology, device 200, where this is not the case. Device 200 is similar to device 100 previously described. Thus, device 200 has a first working end 202 and a second working end 242. The first working end 202 has a first vaned rotor 204 (impeller) and a first anchor 206. The second working end 242 has a second vaned rotor 244 (turbine) and a second anchor 246. Each of these components of device 200 is identical to that of device 100 described hereinabove. Operatively connecting the second vaned rotor 244 with the first vaned rotor 204 is a driveshaft assembly of which only driveshaft 212 is shown. The remainder of the components of the driveshaft assembly are the same as those of device 100 described hereinabove. In this embodiment, the driveshaft 212 has a gearbox 214. The gearbox 214 contains gears that alter the rotation ratio between the second vaned rotor 244 and the first vaned rotor 204, such that the ratio of rotation between them is not 1:1. Different such devices 200 can have different set rotational ratios, with the surgeon selecting the device 200 that has the ratio that is appropriate to the characteristics and conditions of the implantation and operation of the device 200.

In some other embodiments, the gearbox 214 can contain an automatic clutch (to automatically disconnect the first vaned rotor 204 from the rotational movement of the second vaned rotor 244 under particular operating conditions). In yet other embodiments, the gearbox 214 can contain gears that reverse the direction of rotation between the second vaned rotor 244 and the first vaned rotor 204. Finally, in still other embodiments, the gearbox 214 can contain more than one or all of the previously mentioned structures in combination.

### Device Implantation & Explanation

Device 100 can be transcatheterly implanted and explanted using standard conventional techniques. (The WO '765 provides a very detailed description of such techniques, and they are not repeated herein for the sake of brevity.).

For example, in a device 100 to be implanted in a patient to provide renal support (i.e., support for the patient's kidneys), the implantation site of the turbine (i.e., the second implantation site for the second vaned rotor 144) is within supra-renal descending aorta and the implantation site of the impeller (i.e., the first implantation site for the first vaned rotor 104) is within the inferior vena cava. (The device 100 is selected so as to have a drive assembly of the appropriate length.) Thus, at a high level and broadly speaking, the device 100 can be implanted by the surgeon in the following manner by: (1) Obtaining access to the femoral vein of the patient (e.g., via the well-known Seldinger technique). (2) Guiding a guidewire through the vasculature from the access site in the femoral vein to the first implantation site within the inferior vena cava. (3) Using at least in part the guidewire to access the supra-renal descending aorta from the inferior vena cava (e.g., using a standard transcaval technique as used in transcatheter aortic valve implantations (TAVI's)). (4) Guiding the guidewire to the second implantation site within the supra-renal descending aorta. (5) Railing a delivery sheath along the guidewire through the vasculature of the patient to the second implantation site. (6) Advancing the device 100 with the second working end in its delivery configuration and the first working end in its delivery configuration through the delivery sheath second working end first to the second implantation site in the supra-renal descending aorta, leaving the first working end of the device 100 inside the interior vena cava at the first implantation site. (7) Promoting exit of the second working end of the device 100 from the delivery sheath at the second implantation site in the supra-renal descending aorta, e.g., by partially withdrawing the delivery sheath while keeping the device in place; which in this embodiment causes the second working end of the device to adopt its deployed configuration anchoring the second working end of the device 100 in place at the second implantation site within the supra-renal descending aorta. (8) Promoting exit of the first working end of the device 100 from the delivery sheath at the first implantation site in the inferior vena cava; e.g. by partially withdrawing the delivery sheath while keeping the device 100 in place; which in this embodiment causes the first working end of the device 100 to adopt its deployed configuration anchoring the first working end of the device 100 in place at the first implantation site within the inferior vena cava. (9) Withdrawing the delivery sheath from the patient's body. (10) Withdrawing the guidewire from the patient's body.

As a second example, in a device 100 to be implanted in a patient to provide right heart support, the implantation site of the turbine (i.e., the second implantation site for the second vaned rotor 144) is within thoracic descending aorta and the implantation site of the impeller (i.e., the first implantation site for the first vaned rotor 104) is within the pulmonary trunk (or within one of the pulmonary arteries). (The device 100 is selected so as to have a drive assembly of the appropriate length.) Thus, at a high level and broadly speaking, the device 100 can be implanted by the surgeon in the following manner by: (1) Obtaining access to the femoral artery of the patient (e.g., via the well-known Seldinger technique). (2) Guiding a guidewire through the vasculature from the access site in the femoral artery to the second implantation site within the thoracic descending aorta. (3) Using at least in part the guidewire to access the pulmonary trunk from the thoracic descending aorta. (4) Guiding the guidewire to the first implantation site within the pulmonary trunk. (5) Railing a delivery sheath along the guidewire through the vasculature of the patient to the first implantation site. (6) Advancing the device 100 with the first working end in its delivery configuration and the second working end in its delivery configuration through the delivery sheath first working end first to the first implantation site in the pulmonary trunk, leaving the second working end of the device 100 inside the thoracic descending aorta at the second implantation site. (7) Promoting exit of the first working end of the device 100 from the delivery sheath at the first implantation site in the pulmonary trunk, e.g., by partially withdrawing the delivery sheath while keeping the device in place; which in this embodiment causes the first working end of the device to adopt its deployed configuration anchoring the first working end of the device 100 in place at the first implantation site within the pulmonary trunk. (8) Promoting exit of the second working end of the device 100 from the delivery sheath at the second implantation site in the thoracic descending aorta; e.g. by partially withdrawing the delivery sheath while keeping the device 100 in place; which in this embodiment causes the second working end of the device 100 to adopt its deployed configuration anchoring the second working end of the device 100 in place at the second implantation site within the thoracic descending aorta. (9) Withdrawing the delivery sheath from the patient's body. (10) Withdrawing the guidewire from the patient's body.

In both of the above examples, the second vaned rotor of the device may (or may not) be implanted downstream of the outlet of a VAD, if the surgeon so decides for that patient.

Further, in both of the above examples, the device 100 can be explanted by standard retrieval techniques using a snare and a retrieval sheath as described in the WO '765 Publication.

### Operation

In this embodiment, the device 100 simply operates on its own, without any motor or human intervention. The second vaned rotor 144 (the turbine) is caused to rotate by the flow of blood in the aorta (whether that flow has been augmented by an LVAD or not). The rotational movement of the second vaned rotor 144 is transmitted to the first vaned rotor 104 (the impeller) via the driveshaft 112. The first vaned rotor 104 increases the flow rate of the blood in the inferior vena cava or the pulmonary trunk (as the case may be from the above examples).

### Additional Embodiments—Multiple Vaned Rotors

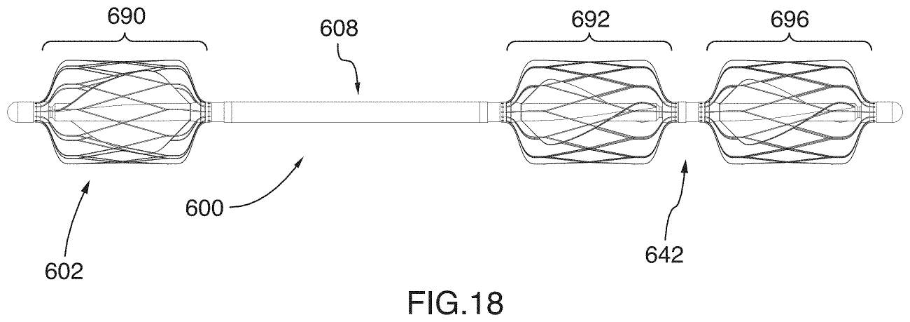

FIGS. 17, 18 & 19, show additional embodiments of the present technology similar to those above, but wherein multiple vaned rotors units (e.g., a vaned rotor and an anchor) are present on one or both of the first working end and the second working end of the device.

In the embodiment shown in FIG. 17, device 500 has a first working end 502, a second working end 542 and a drive shaft assembly 508 operatively interconnecting the first working end 502 and the second working end 542. In this embodiment, the first working end 502 of the device 500 has a first vaned rotor unit 590 and a third vaned rotor unit 594. The second working end 502 of the device 500 has a second vaned rotor unit 592. The driveshaft operatively interconnects each of the vaned rotor units 590, 592, 594.

In the embodiment shown in FIG. 18, device 600 has a first working end 602, a second working end 642 and a drive shaft assembly 608 operatively interconnecting the first working end 602 and the second working end 642. In this embodiment, the first working end 602 of the device 600 has a first vaned rotor unit 690. The second working end 642 of the device 600 has a second vaned rotor unit 692 and a fourth vaned rotor unit 696. There is no third vaned rotor unit in this embodiment. The driveshaft operatively interconnects each of the vaned rotor units 690, 692, 696.

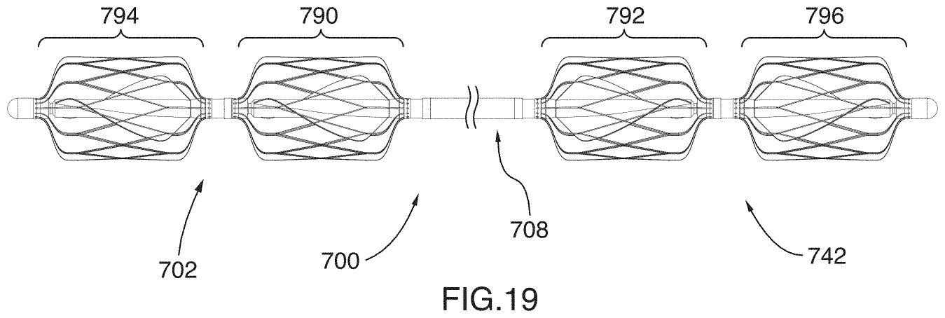

In the embodiment shown in FIG. 19, device 700 has a first working end 702, a second working end 742 and a drive shaft assembly 708 operatively interconnecting the first working end 702 and the second working end 742. In this embodiment, the first working end 702 of the device 700 has a first vaned rotor unit 790 and a third vaned rotor unit 794. The second working end 742 of the device 700 has a second vaned rotor unit 792 and a fourth vaned rotor unit 796. The driveshaft operatively interconnects each of the vaned rotor units 790, 792, 794, 796.

### Additional Embodiments—Device Split into Separate Modules

FIGS. 15 & 16, show an additional embodiment of the present technology wherein the device 400 is “split” into (i.e., designed and manufactured as) two separate modules, a first module 416 and a second module 436. The first module 416 has the first working end 402 of the device 400 and a first portion 408a of the driveshaft assembly. The first working end 402 of device 400 is identical to the first working end 102 of device 100. Thus, the first working end 402 has a first vaned rotor 404 (the impeller) and a first anchor 406. The second module 436 has the second working end 442 of the device 400 and a second portion 408b of the driveshaft assembly. The second working end 442 of device 400 is identical the second working end 142 of device 100. Thus, the second working end 442 has a second vaned rotor 444 (the turbine) and a second anchor 446.

The first portion 408a and the second portion 408b of the driveshaft assembly of device 400 are similar to the driveshaft assembly 108 of device 100. Thus, the first portion 408a of the driveshaft assembly is connected to the first connector 424, which is identical to the first connector 124 of the device 100. The first portion 408a of the driveshaft assembly has a driveshaft 412a which is disposed within the lumen of the protective hollow tubular covering 410a. In this embodiment, the opposite end of the driveshaft 412a of the first module 416 (opposite from the connector 424) has a magnet 418a. Further, the protective hollow tubular covering 410a terminates with a sealed end such that no communication is permitted between the lumen and the external environment of the first module 416.

Similarly, the second portion 408b of the driveshaft assembly is connected to the second connector 454, which is identical to the second connector 154 of the device 100. The second portion 408b of the driveshaft assembly has a driveshaft 412b which is disposed within the lumen of the protective hollow tubular covering 410b. In this embodiment, the opposite end of the driveshaft 412b of the second module 436 (opposite from the connector 454) has a magnet 418b. Further, the protective hollow tubular covering 410b terminates with a sealed end such that no communication is permitted between the lumen and the external environment of the second module 416.

The magnet 418a of the first module 416 and the magnet 418b of the second module 436 are structured to operatively interconnect with each other such that rotational movement of the magnet 418b of the second module 436 is transmitted to the magnet 418a of the first module 416. (Depending on the embodiment and the environment in which the device 400 is implanted, the operative distance between the magnets 418b and 418a varies. Thus, in some embodiments the magnets 418a, 418b physically touch one another whereas in other embodiments the magnets 418a, 418b have a gap between them and remain operatively connected). Thus, in some cases, the first module 416 is dimensioned and shaped to be wholly containable within the first conduit when implanted within the mammalian body and the second module is dimensioned and shaped to be wholly containable within the second conduit when implanted within the mammalian body, with the gap between the magnets 418a, 418b (when the first module 416 and the second module 436 are implanted with the body) being within the operative distance between the magnets 418a, 418b. Thus, it is not necessary to make openings in the walls of the first and second conduits for the device to be implanted, operable, or explanted.

Each of the first and second modules 316, 336 can be transcatheterly implanted and explanted using standard conventional techniques.

As an example, in a device 300 to be implanted in a patient to provide renal support (i.e., support for the patient's kidneys), the implantation site of the turbine (i.e., the second implantation site for the second vaned rotor 144) is within supra-renal descending aorta and the implantation site of the impeller (i.e., the first implantation site for the first vaned rotor 104) is within the inferior vena cava. (The device 300 is selected so that each of the first and the second modules 316, 336 have driveshaft assemblies 108a, 108b of the appropriate length, have sufficient flexibility, and have magnets of appropriate strength.) Thus, at a high level and broadly speaking, the device 300 can be implanted by the surgeon in the following manner by: (1) Obtaining first access to the femoral vein of the patient. (2) Guiding a first guidewire through the vasculature from the access site in the femoral vein to the first implantation site within the inferior vena cava. (3) Railing a first delivery sheath along the first guidewire through the vasculature to the first implantation site within the inferior vena cava. (4) Advancing the first module 316 of the device 300 with the first working end in its delivery configuration through the first delivery sheath the first portion of the driveshaft assembly first to the first implantation site within the inferior vena cava. (5) Promoting exit of the first module 316 of the device 300 from the first delivery sheath at the first implantation site within the inferior vena cava; which in this embodiment causes the first working end of the device 300 to adopt its deployed configuration anchoring the first working end of the device 300 in place within the inferior vena cava. (6) Withdrawing the first delivery sheath from the patient. (7) Withdrawing the first guidewire from the patient. (8) Obtaining second access to the axillary artery of the patient. (9) Guiding a second guidewire through the vasculature from the access site in the axillary artery to the second implantation site within supra-renal descending aorta. (10) Railing a second delivery sheath along the second guidewire through the vasculature to the second implantation site within supra-renal descending aorta. (11) Advancing the second module 336 of the device 300 with the second working end in its delivery configuration through the second delivery sheath the second portion of the driveshaft assembly first to the second implantation site within supra-renal descending aorta. (12) Promoting exit of the second module 336 of the device 300 from the second delivery sheath at the second implantation site within supra-renal descending aorta such that the first magnet 418a and the second magnet 418b form the operative magnetic interconnection with each other without physically crossing walls of the inferior vena cava or the supra-renal descending aorta, which in this embodiment causes the second working end of the device 300 to adopt its deployed configuration anchoring the second working end of the device 300 in place within supra-renal descending aorta. (13) Withdrawing the second delivery sheath from the patient. (14) Withdrawing the second guidewire from the patient.

In the above example, the second vaned rotor of the device may (or may not) be implanted downstream of the outlet of a VAD, if the surgeon so decides for that patient.

Further, the first and second modules 316, 336 can be explanted by standard retrieval techniques using a snare and a retrieval sheath as described in the WO '765 Publication.

### Additional Embodiments—Device with Self-Expanding Sealing Units

In employing some embodiments implementations of the present technology, it may be necessary to seal either one or both of the openings made in the first conduit and the second conduit to allow the driveshaft assembly to pass from the interior of the first conduit (from the first working end) to the interior of the second conduit (to the second working end). Conventional sealing techniques (with which the skilled address would understand) may be used.

Alternatively, in some embodiments, devices of the present technology may include self-expanding sealing units and/or seals, which the surgeon may wish to use.

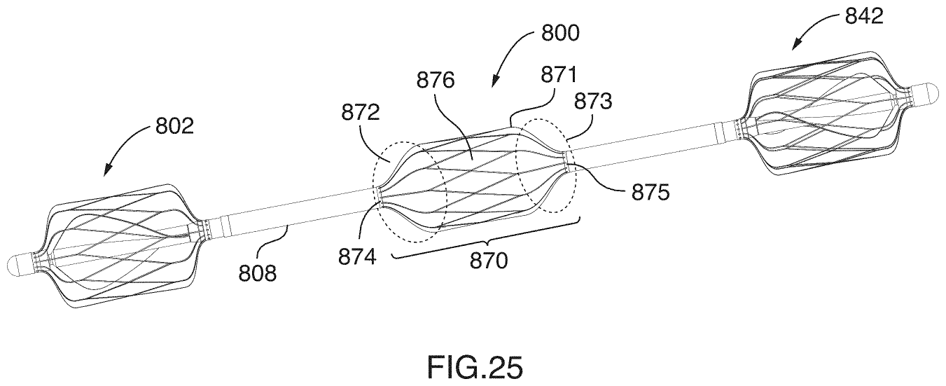

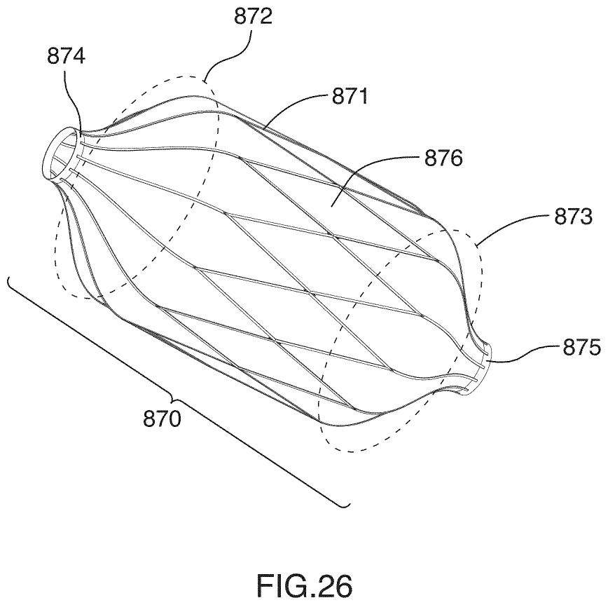

With reference to FIGS. 25 and 26, there is shown a device 800 being a seventh embodiment of the present technology. The device 800 has a first working end 802, a second working end 842, and a driveshaft assembly 808, all of which are identical to their counterparts 102, 142, 108 (as the case may be) of device 100. They thus need not be described in further detail here. In this embodiment, affixed to the outer surface of the protective tubular covering of the driveshaft assembly 108 is a self-expandable sealing unit 870. The sealing unit 870 is a wire network (having wires 871) very similar to the first anchor of the first working end 802 and the second anchor of the second working end 842. At either end of the sealing unit 870 the wires 871 are connected to a band 874, 875 that encircles the driveshaft assembly 808, thus securing the sealing unit 870 in place.

The sealing unit 870 thus has a compact/delivery configuration and an expanded/deployed configuration and is biased towards its expanded/deployed configuration. The sealing unit 870 is in its compact/delivery configuration when in a loader or a catheter (e.g., a delivery sheath, a retrieval sheath, etc.). During implantation of the device 800, as the sealing unit 870 exits the delivery sheath it adopts its expanded/deployed configuration.

The wire network is covered with a medical grade polymeric sheet 876. When the sealing unit is in its expanded/deployed configuration, each of its ends 872, 873 can act as seals to seal openings in the first conduit and second conduit respectively. The surgeon selects the device 800 having a sealing unit 870 having the appropriate dimensions and shape (prior to implantation of the device 800) to accomplish this purpose, and positions the sealing unit 870 appropriately to seal both conduits during device implant.

During explanation of the device 800, as the device 800 enters a retrieval sheath, the sealing unit 870 adopts its compact/delivery configuration, unsealing the openings and allowing for retrieval of the device 800.

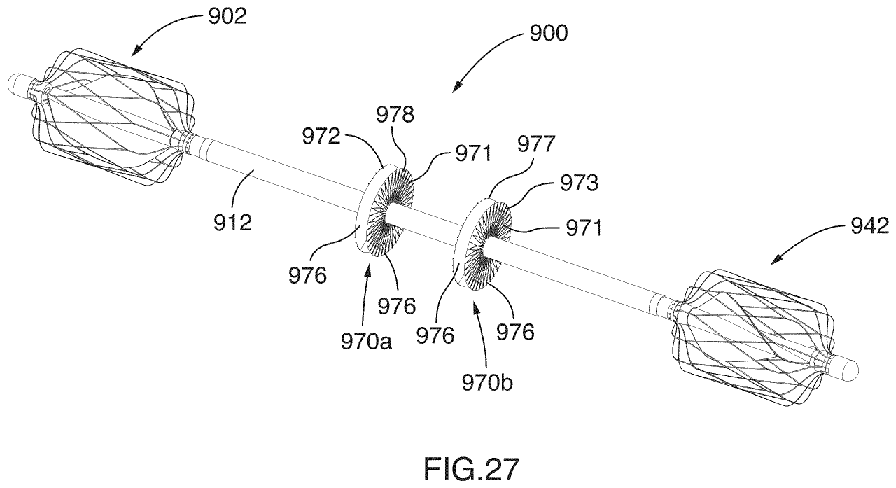

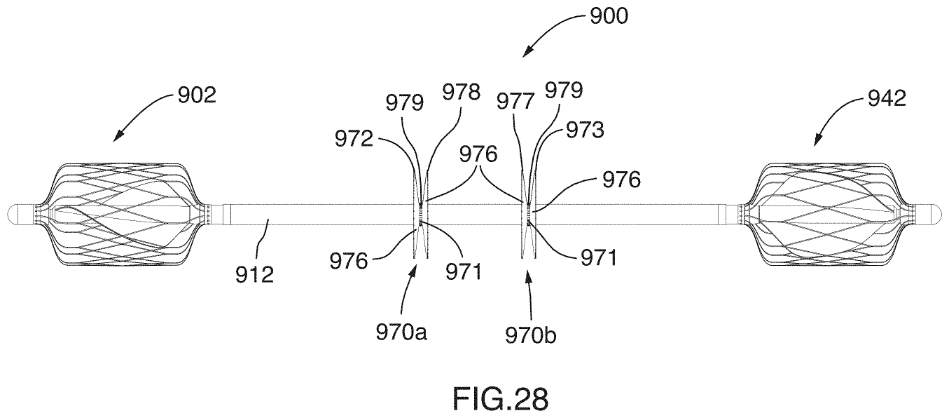

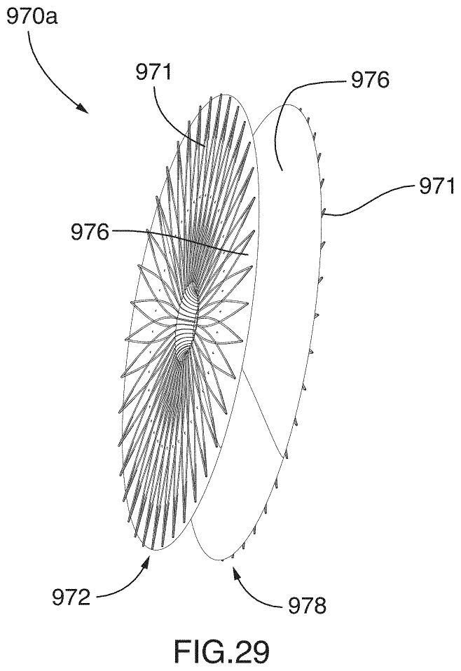

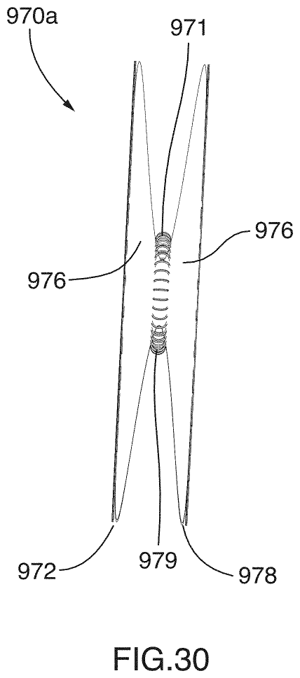

With reference to FIGS. 27 to 30, there is shown a device 900 being an eighth embodiment of the present technology. The device 900 has a first working end 902, a second working end 942, and a driveshaft assembly 908, all of which are identical to their counterparts 102, 142, 108 (as the case may be) of device 100. They thus need not be described in further detail here. In this embodiment, affixed to the outer surface of the protective tubular covering of the driveshaft assembly 908 are two self-expandable sealing units 970a, 970b. Each sealing unit 970a, 970b is wire network (having wires 971) in the shape of two slightly spaced apart radially extending disks. Thus, sealing unit 970a has a first radially extending disk 972 and a second radially extending disk 978, sealing unit 970b has a first radially extending disk 973 and a second radially extending disk 977. (In some embodiments, they may be similar to an Amplatzer™ vascular plug commercialized Abbott™.). As can best be seen in FIG. 30, the wires 971 of sealing unit 970a are connected to a band 979 that encircles the driveshaft assembly 908, thus securing the sealing unit 970a (and its disks 972, 978) in place. Sealing unit 970b is identical to seal unit 970a in this embodiment.

The sealing units 970a, 970b (being of a similar construction as with the first anchor of the first working end 902 and the second anchor of the second working end 942) including their disks 972, 973, 977, 978, thus each have a compact/delivery configuration and an expanded/deployed configuration and are biased towards their expanded/deployed configuration. The sealing units 970a, 970b and their disks 972, 973, 977, 978 are in their compact/delivery configuration when in a loader or a catheter (e.g., a delivery sheath, a retrieval sheath, etc.). During implantation of the device 900, as each disk 972, 973, 977, 978 (as the case may be) of each the sealing unit 970a, 970b exits the delivery sheath that disk adopts its expanded/deployed configuration.

The wire networks of each disk 972, 973, 977, 978 are covered with a medical grade polymeric sheet 976. When a sealing unit 970a, 970b is in its expanded/deployed configuration, each of its disks 972, 973, 977, 978 can act as a seal to seal openings in the first conduit and second conduit (as the case may be). In this particular embodiment, both the interior surface and the exterior surface of each conduit have a disk positioned against them, and thus each sealing unit seals its opening from both sides. Thus, when appropriately positioned, in the expanded/deployed configuration, disk 973 of sealing unit 970b seals the opening in the wall of the second conduit from the inside and disk 977 of sealing unit 970b seals that opening in the wall of the second conduit from the outside. Similarly, when appropriately positioned, in the expanded/deployed configuration, disk 972 of sealing unit 970a seals the opening in the wall of the first conduit from the inside and disk 978 of sealing unit 970a seals that opening in the wall of the first conduit from the outside.

The surgeon selects the device 900 having sealing units 970a, 970b having disks 972, 973, 977, 978 having the appropriate dimensions, shape and locations (prior to implantation of the device 900) to accomplish this purpose, and positions the sealing units 970a, 970b and their disks 972, 973, 977, 978 appropriately to seal both conduits during the device implant.

During explanation of the device 900, as the device 900 enters a retrieval sheath, the sealing units 970a, 970b adopt their compact/delivery configuration, unsealing the openings and allowing for retrieval of the device 900.

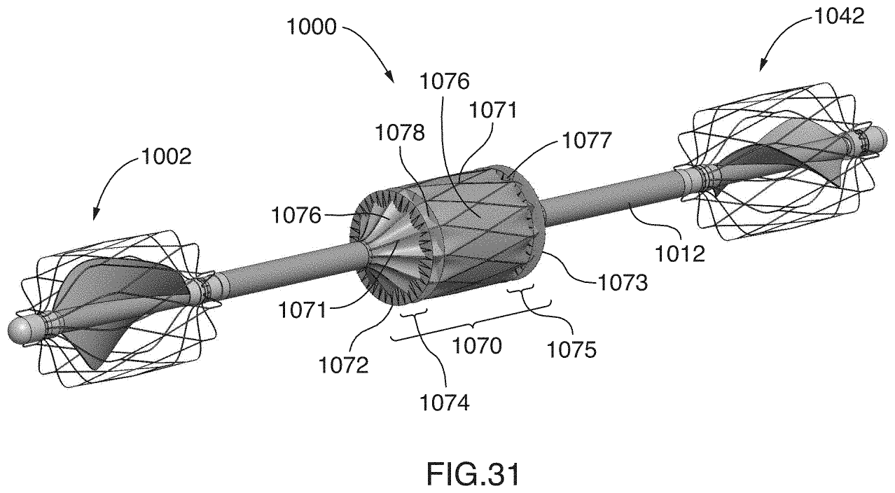

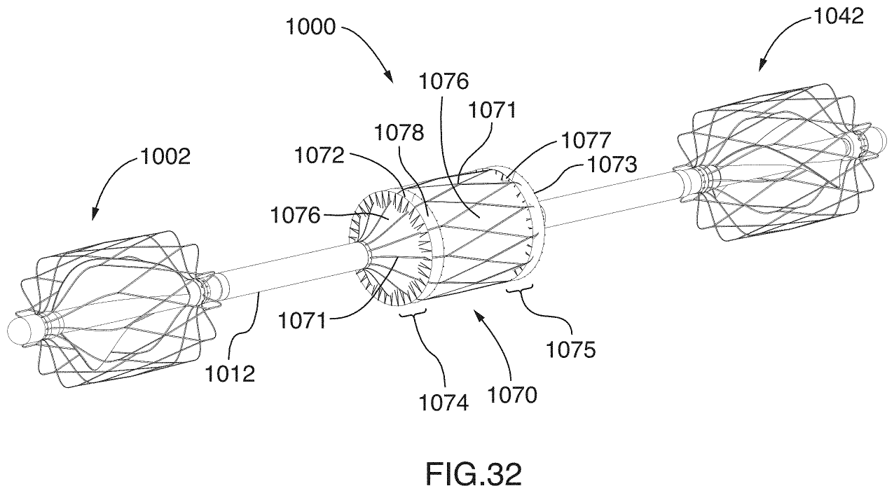

With reference to FIGS. 31 to 32, there is shown a device 1000 being a ninth embodiment of the present technology. The device 1000 has a first working end 1002, a second working end 1042, and a driveshaft assembly 1008, all of which are identical to their counterparts 102, 142, 108 (as the case may be) of device 100. They thus need not be described in further detail here. In this embodiment, affixed to the outer surface of the protective tubular covering of the driveshaft assembly 1008 is a multi-part self-expandable sealing unit 1070 similar in part to the sealing unit 870 of device 800 and in part to the sealing units 970a, 970b of device 900.

In this embodiment, the sealing unit 1070 is wire network (having wires 1071) the central portion of which is in the shape of a large central wire network (similar to sealing unit 870 of device 800) but having two longitudinal ends 1077, 1078 in the shape of radially extending disks. Slightly spaced apart from each longitudinal end 1077, 1078 of the central portion is a radially extending disk 1072, 1073. Disk 1072 is slightly spaced apart from end 1078. Disk 1073 is slight spaced apart from end 1077.

The sealing unit 1070 (being of a similar construction as with the first anchor of the first working end 1002 and the second anchor of the second working end 1042) including its ends 1077, 1078 and the disks 1072, 1073, thus each have a compact/delivery configuration and an expanded/deployed configuration and are biased towards their expanded/deployed configuration. The sealing unit 1070 is in its compact/delivery configuration when in a loader or a catheter (e.g., a delivery sheath, a retrieval sheath, etc.). During implantation of the device 1000, the sealing unit 1070 including its ends 1077, 1078 and the disks 1072, 1073 as they exit the delivery sheath adopt their expanded/deployed configuration.

The wire network of the sealing unit 1070 is covered with a medical grade polymeric sheet 1076. When sealing unit 1070 is in its expanded/deployed configuration, each of its ends 1077, 1078 and each of its disks 1072, 1073 can act as a seal to seal openings in the first conduit and second conduit (as the case may be). In this particular embodiment, both the interior surface and the exterior surface of each conduit have one of an end 1077, 1078 or a disk 1072, 1073 positioned against them, and thus the sealing unit 1070 seals the openings in both conduits from both sides. Thus, when appropriately positioned, in the expanded/deployed configuration, disk 1073 of sealing unit 1070 seals the opening in the wall of the second conduit from the inside and end 1077 of sealing unit 1070 seals that opening in the wall of the second conduit from the outside. Similarly, when appropriately positioned, in the expanded/deployed configuration, disk 1072 of sealing unit 1070 seals the opening in the wall of the first conduit from the inside and end 1078 of sealing unit 1070 seals that opening in the wall of the first conduit from the outside.

The surgeon selects the device 100 having a sealing unit 1070 with component parts having the appropriate dimensions, shape and locations (prior to implantation of the device 1000) to accomplish this purpose, and positions the central portion (and particularly the ends 1077, 1078 thereof) and the disks 1072, 1073 appropriately to seal both conduits during the device implant.

During explanation of the device 1000, as the device 1000 enters a retrieval sheath, the sealing unit 1070 (including its various component parts) adopt their compact/delivery configuration, unsealing the openings and allowing for retrieval of the device 1000.

### ADDITIONAL INFORMATION & INCORPORATIONS-BY-REFERENCE

As a skilled addressee would understand, percutaneously transcatheterly implantable intralumenal blood pumps (device to which the device 100 of the present technology is related) are well known in the art. Thus, for purposes of brevity, no attempt has been made herein to describe many conventional details with which the skilled addressee would be familiar. However, to facilitate understanding of such devices (e.g., by readers not skilled in the art), reference may be had to one or more of the following patent documents, which are incorporated herein by reference in their entirety for all purposes:
United States Patent Application Publication No. US 2020/0405926 A1 (Alexander et al.), published Dec. 31, 2020, entitled “Removable Mechanical Circulatory Support for Short Term Use”; and
U.S. Pat. No. 10,722,631 B2 (Salahieh et al.), issued Jul. 29, 2020, entitled

“Intravascular Blood Pumps and Methods of Use and Manufacture”.

The above list is not intended to be a complete list for any purpose. It is only intended to provide some examples of some documents believed to be potentially useful. Percutaneously transcatheterly implantable intravascular blood pumps have been described in the literature at least since the 1980's, and thus there are many documents that might be helpful that are not set forth above.

In addition, the following patent document commonly owned by the assignee of the present application is also incorporated herein by reference in its entirety for all purposes. This document may also provide additional background, especially to the unskilled reader:
Int'l Pat. App. No. PCT/US2021/012083 (Puzzle Medical Devices Inc, et al.), filed Jan. 4, 2021, entitled “Mammalian Body Conduit Intralumenal Device and Lumen Wall Anchor Assembly, Components Thereof and Methods of Implantation and Explantation Thereof”.

### MISCELLANEOUS

The present technology is not limited in its application to the details of construction and the arrangement of components set forth in the preceding description or illustrated in the drawings. The present technology is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the description the same numerical references refer to similar elements.

It must be noted that, as used in this specification and the appended claims, the singular form “a”, “an” and “the” include plural referents unless the context clearly dictates otherwise.

As used herein, the term “about” or “generally” or the like in the context of a given value or range (whether direct or indirect, e.g., “generally in line”, “generally aligned”, “generally parallel”, etc.) refers to a value or range that is within 20%, preferably within 10%, and more preferably within 5% of the given value or range.

As used herein, the term “and/or” is to be taken as specific disclosure of each of the two 10 specified features or components with or without the other. For example, “A and/or B” is to be taken as specific disclosure of each of (i) A, (ii) B and (iii) A and B, just as if each is set out individually herein.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1. A mammalian body intralumenal implantable fluid flow influencing device for influencing flow of a first fluid within a first conduit of the mammalian body via a flow of a second fluid within a second conduit of the mammalian body,
the device comprising:
a first working end, the first working end having a first vaned rotor and a first anchor for anchoring the first working end within the first conduit, the first working end having a delivery configuration in which it is dimensioned and shaped to be delivered to a first implantation site within the first conduit and having a deployed configuration,
a second working end, the second working end having a second vaned rotor and a second anchor for anchoring the second working end within the second conduit, the second working end having a delivery configuration in which it is dimensioned and shaped to be delivered to a second implantation site within the second conduit and having a deployed configuration, and
a driveshaft assembly operatively interconnecting the second vaned rotor with the first vaned rotor to transmit rotational movement of the second vaned rotor to the first vaned rotor;
the device being motorless; and
when the device is implanted within the mammalian body,
the second vaned rotor acts as a turbine and is caused to rotate by the flow of the second fluid within the second conduit, thereby extracting kinetic energy from the second fluid, and
the first vaned rotor acts an impeller and is caused to rotate as a result of the rotational movement of the second vaned rotor, thereby imparting kinetic energy to the first fluid.

2. The device of claim 1, wherein
the first anchor includes a first wire network, the first wire network having a compact configuration and an expanded configuration, the first wire network being in its compact configuration when the first working end is in its delivery configuration, the first wire network being in its expanded configuration when the first working end is in its deployed configuration; and
the second anchor includes a second wire network, the second wire network having a compact configuration and an expanded configuration, the second wire network being in its compact configuration when the second working end is in its delivery configuration, the second wire network being in its expanded configuration when the second working end is in its deployed configuration.

3. The device of claim 2, wherein:
when the first wire network is in its expanded configuration, the first wire network surrounds the first vaned rotor and exerts a force on the first conduit sufficient to anchor the first working end in place when the device is in operation; and
when the second wire network is in its expanded configuration, the second wire network surrounds the second vaned rotor and exerts a force on the second conduit sufficient to anchor the second working end in place when the device is in operation.

4. A method of implanting a motorless intralumenal implantable fluid flow influencing device within a mammalian body, the device having,
a first working end, the first working end having a first vaned rotor and a first anchor for anchoring the first working end within the first conduit, the first working end having a delivery configuration in which it is dimensioned and shaped to be delivered to a first implantation site within the first conduit and having a deployed configuration,
a second working end, the second working end having a second vaned rotor and a second anchor for anchoring the second working end within the second conduit, the second working end having a delivery configuration in which it is dimensioned and shaped to be delivered to a second implantation site within the second conduit and having a deployed configuration, and
a driveshaft assembly operatively interconnecting the second vaned rotor with the first rotor to transmit rotational movement of the second vaned rotor to the first vaned rotor,
the method comprising:
a) obtaining access to a conduit system of the mammalian body of which the first conduit is a part;
b) guiding a guidewire through the conduit system to the first implantation site within first conduit;
c) using at least in part the guidewire to access the second conduit from the first conduit;
d) guiding the guidewire to the second implantation site within the second conduit;
e) railing a delivery sheath along the guidewire through the conduit system, the first conduit, and the second conduit, to the second implantation site;
f) advancing the device with the second working end in its delivery configuration and the first working end in its delivery configuration through the delivery sheath second working end first to the second implantation site leaving the first working end of the device inside the first conduit;
g) promoting exit of the second working end of the device from the delivery sheath at the second implantation site;
h) causing the second working end of the device to adopt its deployed configuration anchoring the second working end of the device in place within the second conduit;
i) promoting exit of the first working end of the device from the delivery sheath at the first implantation site;
j) causing the first working end of the device to adopt its deployed configuration anchoring the first working end of the device in place within the first conduit;
k) withdrawing the delivery sheath from the mammalian body; and
l) withdrawing the guidewire from the mammalian body.

5. The method of claim 4, wherein the second implantation site is downstream from an outlet of a powered ventricular assist device.

6. A method of implanting a motorless intralumenal implantable fluid flow influencing device within a mammalian body, the device having,
a first working end, the first working end having a first vaned rotor and a first anchor for anchoring the first working end within the first conduit, the first working end having a delivery configuration in which it is dimensioned and shaped to be delivered to a first implantation site within the first conduit and having a deployed configuration,
a second working end, the second working end having a second vaned rotor and a second anchor for anchoring the second working end within the second conduit, the second working end having a delivery configuration in which it is dimensioned and shaped to be delivered to a second implantation site within the second conduit and having a deployed configuration,
a driveshaft assembly operatively interconnecting the second vaned rotor with the first rotor to transmit rotational movement of the second vaned rotor to the first vaned rotor,
a first module,
including the first working end and a first portion of the driveshaft assembly, the first portion of the driveshaft assembly having a first connector having a first magnet, and
being dimensioned and shaped to be wholly containable within the first conduit when implanted within the mammalian body, and
a second module,
including the second working end and a second portion of the driveshaft assembly, the second portion of the driveshaft assembly having a second connector having a second magnet, the first connector and the second connector each being structured to form an operative magnetic interconnection with the other without physically crossing walls of the first conduit and the second conduit, and
being dimensioned and shaped to be wholly containable within the second conduit when implanted within the mammalian body,
the method comprising:
a) obtaining first access to a conduit system of the mammalian body of which the first conduit is a part;
b) guiding a first guidewire through the conduit system to the first implantation site within first conduit;
c) railing a first delivery sheath along the first guidewire through the conduit system and the first conduit to the first implantation site;
d) advancing the first module of the device with the first working end in its delivery configuration through the first delivery sheath the first portion of the driveshaft assembly first to the first implantation site;
e) promoting exit of the first module of the device from the first delivery sheath at the first implantation site;
f) causing the first working end of the device to adopt its deployed configuration anchoring the first working end of the device in place within the first conduit;
g) withdrawing the first delivery sheath from the mammalian body;
h) withdrawing the first guidewire from the mammalian body;
i) obtaining second access to a conduit system of the mammalian body of which the second conduit is a part;
j) guiding a second guidewire through the conduit system to the second implantation site within second conduit;
k) railing a second delivery sheath along the second guidewire through the conduit system and the second conduit to the second implantation site;
l) advancing the second module of the device with the second working end in its delivery configuration through the second delivery sheath the second portion of the driveshaft assembly first to the second implantation site;
m) promoting exit of the second module of the device from the second delivery sheath at the second implantation site such that the first connector and the second connector form the operative magnetic interconnection with each other without physically crossing walls of the first conduit and the second conduit;
n) causing the second working end of the device to adopt its deployed configuration anchoring the second working end of the device in place within the second conduit;
o) withdrawing the second delivery sheath from the mammalian body; and
p) withdrawing the second guidewire from the mammalian body.

7. The method of claim 6, wherein the second implantation site is downstream from an outlet of a powered ventricular assist device.
